(19) United States
(12) Patent Application Publication
(10) Pub. No.: US 2026/0240751 A1
(43) Pub. Date: Aug. 20, 2026

(54) COSMETIC OR DERMATOLOGICAL COMPOSITION COMPRISING POLYLYSINE DENDRIMERS AND USE THEREOF

(71) Applicant: Abdel Hafid Belhadj-Tahar, Toulouse (FR)

(72) Inventor: Abdel Hafid Belhadj-Tahar, Toulouse (FR)

(21) Appl. No.: 18/876,599

(22) PCT Filed: Jun. 16, 2023
(86) PCT No.: PCT/FR2023/050883

§ 371 Date: Dec. 18, 2024

(30) Foreign Application Priority Data
Jun. 20, 2022 (WO) PCT/FR2022/051194

(51) Int. Cl. A61K 8/88; A61K 8/67; A61K 31/07; A61K 31/375; A61K 31/785; A61P 17/00; A61P 17/02; A61P 17/12; A61Q 19/00

(52) U.S. Cl. CPC A61K 8/88; A61K 8/671; A61K 8/675; A61K 8/676; A61K 31/07; A61K 31/375; A61K 31/785; A61P 17/00; A61P 17/02; A61P 17/12; A61Q 19/00; A61Q 19/007; A61K 2800/10

(57) ABSTRACT

The present invention relates to a cosmetic or dermatological composition comprising at least one substituted or unsubstituted polylysine dendrimer, at least one vitamin and at least one cosmetically and/or dermatologically acceptable carrier. The present invention further relates to a non-therapeutic cosmetic care method, comprising applying, on the skin or the mucous membranes, a cosmetic composition comprising at least one substituted or unsubstituted polylysine dendrimer, at least one vitamin and at least one cosmetically and/or dermatologically acceptable carrier. The present invention further relates to the use of the dermatological composition in the treatment of a skin condition.

### TECHNICAL FIELD

The present invention relates to a cosmetic or dermatological composition comprising at least one substituted or unsubstituted polylysine dendrimer, at least one vitamin and at least one cosmetically and/or dermatologically acceptable carrier.

The present invention also relates to cosmetic use for action on the skin or mucous membranes.

The present invention further relates to the use of the dermatological composition in the treatment of a skin condition.

The present invention particularly applies to the field of cosmetics and/or dermatology.

### STATE OF THE ART

The skin is one of the most complex organs in the human body, and the surface area thereof varies according to the size and weight of the subject. The skin represents approximately 2 m² of surface area per individual.

The skin is made up of three very distinct layers, with associated appendages, such as sweat glands and pilosebaceous follicles.

More precisely, from the outside inwards, it consists of:
the epidermis comprising the stratum corneum which is the outermost cellular layer of the skin and which plays an essential role in the skin due to its structure and its contact with the external environment. It is the main barrier of the skin to external aggressors, for example physical (UV rays, etc.), chemical (organic solvents, acids, bases, etc.) and mechanical (cuts, pinching, etc.) aggressors,
the dermis which is a fibro-elastic connective tissue composed of fibroblasts and immune cells (macrophages, lymphocytes, eosinophils) and an intercellular matrix. This matrix is made up of fibrous proteins (collagen, elastin, reticulin) and an interfibrillar gel composed of proteoglycans, glycoproteins, salts and water. The dermis also comprises extensive vascular branches, nerve fibers and skin appendages. It ensures skin suppleness, hydration, nutrition and elasticity.
the hypodermis which is a loose connective tissue with a similar structure to that of the dermis with a predominance of collagen fibers, proteoglycans and tissue. It isolates the dermis from the fibrous membranes enveloping deep underlying parts such as the muscles.

Each of the elements making up the skin has a different or complementary function and metabolism, in close relation to their architecture and their surrounding environment. Their genetic or acquired metabolic dysfunctions linked to age and/or to the environment lead to physio-pathological consequences that cause skin and subcutaneous cell tissue diseases, aging and skin degeneration.

Vitamins and metabolites thereof are known to have a time- and dose-dependent effect on epithelial cell proliferation and differentiation, epithelial and mesenchymal cell synthesis ability, immune modulation and protection against cancer and other diseases, including autoimmune and infectious diseases.

There are cosmetic products and/or compositions comprising vitamins and derivatives thereof that can be applied to the skin in order to restore and protect it. These notably include cosmetic products, in particular compositions for topical use, comprising vitamins and derivatives thereof, for example vitamin A, in particular retinol and derivatives thereof, vitamin C, in particular ascorbic acid and derivatives thereof and/or vitamin E, in particular tocopherol. These compositions may be used in anti-aging, antioxidant or anti-inflammatory treatments.

However, these compositions have limited efficacy, particularly in the skin, for example in anti-aging, anti-inflammatory and/or antioxidant treatments, due in particular to the low bioavailability of the vitamins present in known compositions. In particular, the efficacy of known compositions comprising vitamins is considered by users to be low, notably due to the virtual non-penetration of said vitamins into skin tissue.

There is therefore a real need to find new compositions with good cosmetic or dermatological efficacy, allowing for example the appearance and surface qualities of the skin to be improved, particularly for moisturizing, anti-aging, anti-inflammatory and/or antioxidant effects.

There are also cosmetic compositions comprising vitamins, in particular vitamin A, which are effective in anti-aging cosmetic treatments, for example by reducing the appearance of wrinkles and/or improving the evenness of skin tone and/or increasing the firmness and elasticity of the skin. However, these compositions can induce undesirable side effects, for example linked to an inflammatory reaction and/or skin intolerance and/or skin irritation.

There is therefore a real need to find new cosmetic or dermatological compositions having cosmetic and/or dermatological efficacy that do not induce undesirable side effects, for example skin inflammation and/or skin irritation and/or skin allergy.

There are also cosmetic compositions comprising preservatives, for example benzoic acid and salts thereof, benzyl alcohol, salicylic acid and salts thereof, sorbic acid and salts thereof, dehydroacetic acid and salts thereof, notably used to prevent the growth of micro-organisms, for example bacteria and/or fungi in cosmetic compositions. However, these compounds can induce undesirable side effects when using said compositions, for example an inflammatory reaction and/or skin intolerance and/or skin irritation.

Document CN108743452 discloses a method for preparing cosmetic compositions comprising, in particular, crosslinked and hyperbranched dendritic polylysines which are obtained by a cross-linking reaction of lysine hydrochloride by thermal polymerization in the presence of a caustic alkali. The synthesis of the dendritic polylysines used in this document is an uncontrolled synthesis notably described in document (Scholl, Markus, et al. “The thermal polymerization of amino acids revisited; Synthesis and structural characterization of hyperbranched polymers from l-lysine.” Journal of Polymer Science Part A: Polymer Chemistry 45.23 (2007): 5494-5508 [10]), the polylysine polymer thus obtained and used in this document is poorly structured, polydisperse and hyperbranched. This document also mentions the use of compositions comprising said crosslinked and hyperbranched dendritic polylysines for skin care but demonstrates no significant difference with respect to the cosmetic effect, for example concerning skin elasticity and/or hydration, between the use of a composition comprising or not comprising crosslinked and hyperbranched dendritic polylysines.

There is therefore a real need to find new compositions with good cosmetic or dermatological efficacy, making it possible, for example, to improve the appearance and surface qualities of the skin, particularly for moisturizing, anti-aging, anti-inflammatory and/or antioxidant effects, and/or without inducing undesirable side effects.

### DESCRIPTION OF THE INVENTION

The present invention is precisely designed to meet these needs and drawbacks of the prior art.

The inventor is the very first to combine at least one polylysine dendrimer and at least one vitamin to specifically meet the above-mentioned needs.

The inventors of the present application have highlighted properties of the combination that had never been described or suggested in the prior art.

Surprisingly and unexpectedly, the inventors have particularly demonstrated that the combination of at least one polylysine dendrimer and at least one vitamin advantageously improves the appearance and surface qualities of the skin, particularly for anti-aging and soothing effects.

Surprisingly and unexpectedly, the inventors have particularly demonstrated that a cosmetic composition comprising at least one polylysine dendrimer and at least one vitamin advantageously increases skin hydration, in particular advantageously allows a rapid increase in skin hydration, and advantageously a long-term hydrating effect, for example over a period of at least 28 days.

Surprisingly and unexpectedly, the inventors have particularly demonstrated in particular that a cosmetic composition comprising at least one polylysine dendrimer and at least one vitamin advantageously increases skin hydration, for example via a nourishing effect on the skin, reduces skin tightness and/or increases skin suppleness.

The inventor has also demonstrated that the combination of at least one polylysine dendrimer and at least one vitamin advantageously increases skin hydration, for example by reducing skin tightness, increasing skin suppleness and increasing skin nourishment.

The inventor has also demonstrated that the combination of at least one polylysine dendrimer and at least one vitamin advantageously improves the appearance of the skin, for example in the case of eczema-type lesions, and advantageously reduces itching, particularly of the scalp.

The inventor has also demonstrated that the combination of at least one polylysine dendrimer and at least one vitamin advantageously modifies the release of vitamins present in the composition, accelerates the dissolution of vitamins advantageously allowing an increase in the percutaneous absorption rate of vitamins and a vectorization of vitamins on the skin via the follicular pathway.

The inventor has also surprisingly demonstrated that the combination of at least one polylysine dendrimer and at least one vitamin offers a very high skin tolerance with respect to known compositions comprising vitamins in particular, advantageously making it possible to avoid the use of topical antiseptic compounds and antibiotics, for example when used in the treatment of skin conditions.

As used herein, “subject” means any animal, for example, a mammal, including, but not limited to, humans, non-human primates, rodents and the like, which is to be the recipient of special treatment. Typically, the terms “subject” and “patient” are used interchangeably herein to refer to a human subject

A first object of the invention relates to a cosmetic or dermatological composition comprising at least one substituted or unsubstituted polylysine dendrimer, at least one vitamin and at least one cosmetically and/or dermatologically acceptable carrier.

Polylysine is used herein to mean any polymer wherein the majority unit element is lysine.

Dendrimers are used herein to mean synthetic polymers characterized by repetitive chain branching emanating from a central core, giving rise to a fractal-like topology and a large number of chain ends. Dendrimers are composed of a core, one or more layers (or generations) of branched monomers and a layer of end groups that increase with each “generation” and terminate the various chains. For example, a first-generation polylysine dendrimer (G1) may comprise 8 branched monomers, a second-generation polylysine dendrimer (G2) may comprise 48 branched monomers, a third-generation polylysine dendrimer (G3) may comprise 123 branched monomers, a fourth-generation polylysine dendrimer (G4) may comprise 365 branched monomers, a fifth-generation polylysine dendrimer (G5) may comprise 963 monomers.

According to the invention, the polylysine dendrimer may be any suitable polylysine dendrimer known to the skilled person. An example is a poly-L-lysine dendrimer (DGL) comprising L-lysine monomers associated in a tree-like process around a multifunctional central core. An example is a polylysine dendrimer as described in patent WO2015/104589.

For example, the dendrimer according to the invention may be represented according to formula (I) as follows:

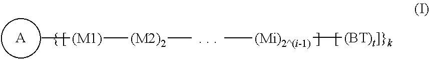

wherein:
(a) A represents the core of the dendrimer, of multivalence k, where:
   k represents the number of dendrons and is preferably equal to 3;
   A represents a core synthon having the structure of the following formula (II)

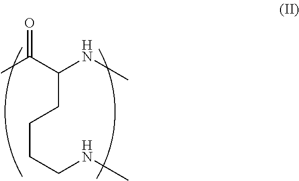

or
of formula (III)

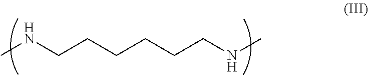

(b) Mi represents a monomer of generation i, where:
   i is an integer from 2 to G, G being the generation number of the dendrimer;
   when i=0, Mi represents: ø, and the terminal branch BT is then directly linked to the base synthon A;
   when i>0, Mi represents the structure of formula (IV):

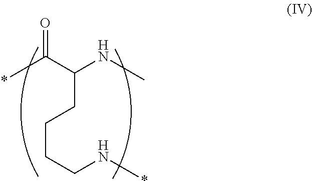

or
of formula (V)

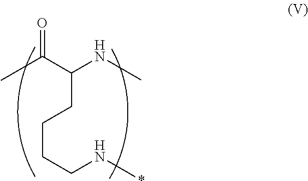

where the symbol * designates the point of attachment of monomer Mi with the monomer of the next generation;
(c) BT represents a terminal branch, and t the number of terminal fragments where:
t is an integer from 1 to 3, preferably t is 2 or 3;
each occurrence of BT independently represents a hydrogen atom, a carboxyl fragment (COOH), a carboxylate ester (COO—R), a hydroxyl group (OH); a thiol group (SH) or a thiol ester fragment (S—R) wherein each occurrence of R independently represents a C1-C6 alkyl or C6 aryl group.

Generation G can range from 2 to 10. For example, G may be equal to 2, 3, 4 or 5; preferably, G may be equal to 2 or 3.

This may be, for example, a poly-L-lysine dendrimer of the following formula (VI):

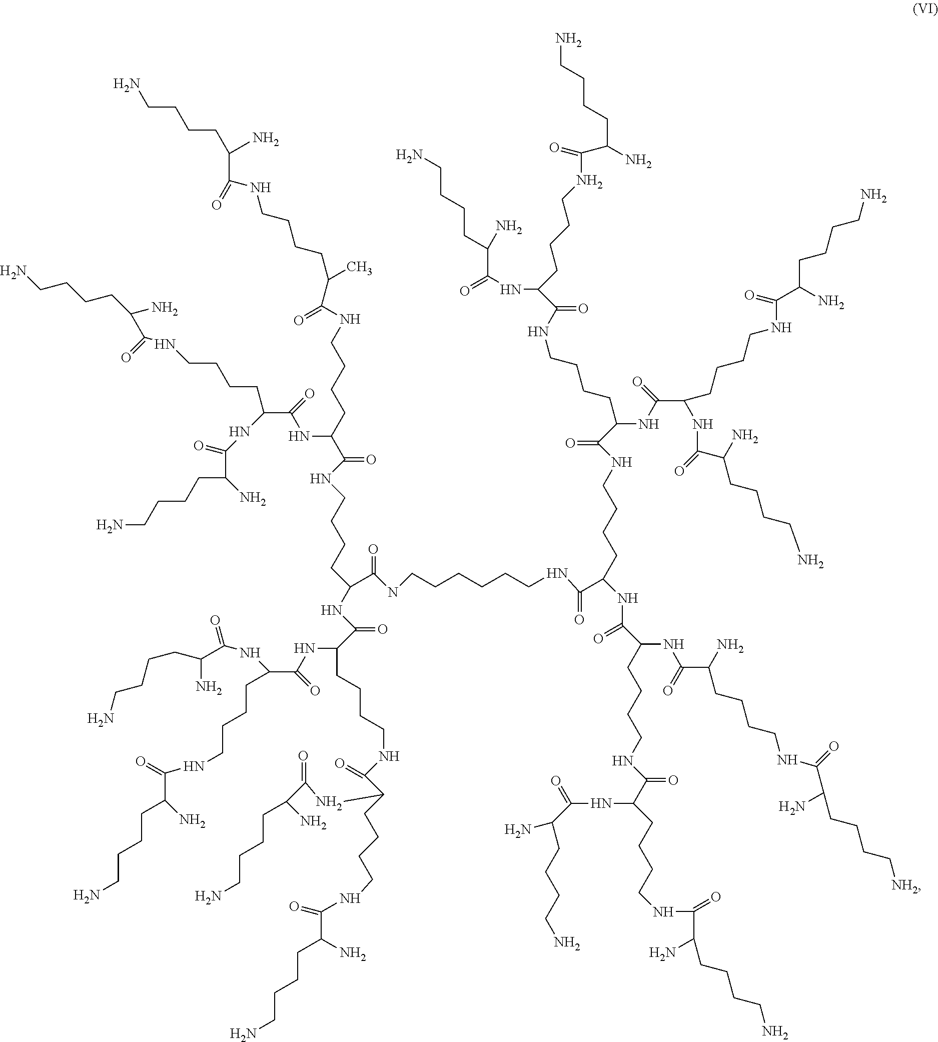 [SMILES: CC(CCCCNC(=O)C(N)CCCCN)C(=O)NCCCCC(NC(=O)C(CCCCNC(=O)C(N)CCCCN)NC(=O)C(N)CCCCN)C(=O)NCCCCC(NC(=O)C(CCCCNC(=O)C(CCCCNC(=O)C(N)CCCCN)NC(=O)C(N)CCCCN)NC(=O)C(CCCCNC(=O)C(N)CCCCN)NC(=O)C(N)CCCCN)C(=O)NCCCCCCNC(=O)C(CCCCNC(=O)C(CCCCNC(=O)C(CCCCNC(=O)C(N)CCCCN)NC(=O)C(N)CCCCN)NC(=O)C(CCCCNC(=O)C(N)CCCCN)NC(=O)C(N)CCCCN)NC(=O)C(CCCCNC(=O)C(CCCCNC(=O)C(N)CCCCN)NC(=O)C(N)CCCCN)NC(=O)C(N)CCCCNC(=O)C(N)CCCCN]

or
a poly-L-lysine dendrimer of formula (VII)
or a poly-L-lysine dendrimer of formula (VIII)

The poly-L-lysine dendrimer of formula (VIII) is also shown in FIG. 8.

In the present by generation, G means the number of layers in the dendrimer, and Z is the number of terminal groups on the outer surface of the dendrimer. As used herein, the core is generation 0 (GO), a monomer directly attached to the core can be considered a 1st generation monomer (G1); a monomer attached to a G1 monomer is a 2nd generation monomer (G2), etc. In this numbering system, 2 (G+1) is used. In this numbering system 2 (G+1), thus, for a dendrimer whose core has 2 functional groups, for G0 2 (2 (0+1), for G1 4 (2 (1+1), etc.

Advantageously, the dendrimer according to the invention has a generation value of at least 2, the core being assigned generation zero.

According to the invention, the substituted or unsubstituted polylysine dendrimer may be a 2nd generation (G2) to 10th generation (G10) polylysine dendrimer. For example, the dendrimer may have a generation value of 2, 3, 4, 5, 6, 7, 8, 9 or 10. Advantageously, the dendrimer may have a generation value of 2, 3, 4, 5, 6, 7; preferably 2, 3.

It may be for example a polylysine dendrimer comprising 48 to 963 lysines, for example a polylysine dendrimer comprising 48 to 123 lysines, for example a polylysine dendrimer comprising 48 lysines or a polylysine dendrimer comprising 123 lysines.

It may be for example a polylysine dendrimer having an average molecular weight of 11 to 250 kDa, for example 11.80 to 249.80 kDa, for example a polylysine dendrimer having an average molecular weight of 11.80 to 32.10 kDa, for example a polylysine dendrimer having an average molecular weight of 11.80 to 32.10 kDa.

It may be for example a polylysine dendrimer with a generation value of 2, comprising for example 48 lysines and having a molecular weight of 11.80 kDa or a polylysine dendrimer with a generation value of 3, comprising for example 123 lysines and having an average molecular weight of 32.10 kDa.

According to the invention, the polylysine dendrimer may be substituted or unsubstituted.

According to the invention, when the polylysine dendrimer is unsubstituted, it may comprise at these ends a cationic ammonium group of the lysine and optionally a counterion. According to the invention, the counterion may be, for example, a Tri-Fluoroacetic acid or TFA group, Acetate or C₂H₅OO⁻ or Chloride Cl⁻.

According to the invention, when the polylysine dendrimer is substituted, it may be substituted with at least one group selected from aspartate, succinate, guanidyl, acetate or fluorine. Preferably, the polylysine dendrimer is substituted with an acetate group.

According to the invention, the polylysine dendrimer, for example poly-L-lysine, may be a commercially available dendrimer. It may be for example the poly-L-lysine dendrimer marketed by COLCOM under the trade name COL-DGL2-NH2 or Dendri Graft Poly-L-Lysines-Generation 2-Amino surface with the molecular formula [H(C₆H₁₃N₂O+)₄₈OH](CF₃CO₂⁻)₄₈ and/or poly-L-lysine dendrimer marketed by COLCOM under the trade name COL-DGL3-NH2 or Dendri Graft Poly-L-Lysines-Generation 3-Amino surface with molecular formula [H(C₆H₁₃N₂O+)₁₂₃OH](CH₃CO₂⁻)₁₂₃. It may be for example a poly-L-lysine dendrimer obtained according to the method described in WO2006114528A1 and/or in the bibliographic reference An Expeditious Multigram-Scale Synthesis of Lysine Dendrigraft (DGL) Polymers by Aqueous N-Carboxyanhydride Polycondensation. H. Collet and collaborators: Chem. Eur. J. 2010, 16, 2309-2316 [2].

Advantageously, the dendrimer is monodisperse and of known uniform spherical structure with a well-defined diameter, for example for the polylysine dendrimer with a generation value of 2: a diameter of 4.5 nm, for example for the polylysine dendrimer with a generation value of 3: a diameter of 7 nm.

Advantageously, the polylysine dendrimer has an estimated free volume of 80%. Advantageously, the polylysine dendrimer allows vectorization of small molecules, for example having a diameter of 4.5 to 8.5 nm, for example at least one vitamin.

Advantageously, the poly-L-lysine dendrimer is obtained according to the method described in WO2006114528A1 and/or in the bibliographic reference An Expeditious Multigram-Scale Synthesis of Lysine Dendrigraft (DGL) Polymers by Aqueous N-Carboxyanhydride Polycondensation. H. Collet and collaborators: Chem. Eur. J. 2010, 16, 2309-2316 [2].

The composition of the invention may comprise from 0.01 to 10% by weight of substituted or unsubstituted polylysine dendrimer, with respect to the total weight of the composition. For example, the composition may comprise from 0.02 to 5%, from 0.02 to 3%, from 0.02 to 0.05% by weight of substituted or unsubstituted polylysine dendrimer with respect to the total weight of the composition.

According to the invention, a vitamin means any suitable vitamin known to the skilled person and/or commercially available. It may be for example a vitamin selected from the group comprising water-soluble vitamins and fat-soluble vitamins.

According to the invention, water-soluble vitamin means a vitamin selected from the group comprising vitamin C, vitamin B1, vitamin B2, vitamin B3, vitamin B5, vitamin B8, vitamin B9, vitamin B12

According to the invention, fat-soluble vitamin means a vitamin selected from the group comprising vitamin A, vitamin D, vitamin E, vitamin K1 and vitamin K2.

According to the invention, the vitamin may be selected from the group comprising vitamin B1, vitamin B2, vitamin B3, vitamin B5, vitamin B8, vitamin B9, vitamin B12, vitamin C, vitamin A, vitamin D, vitamin E, vitamin K1 and vitamin K2, preferably selected from the group comprising vitamin C and vitamin A.

As used herein, the vitamin may be vitamin C (L-ascorbic acid [CAS-No.: 50-81-7]) marketed by Sigma-Aldrich laboratories, vitamin A (retinyl acetate) [CAS-No.: 127-47-9] marketed by Sigma-Aldrich Laboratories, France.

Advantageously, when the composition comprises vitamin C, the composition may advantageously have, for example when applied to the skin, a soothing effect, for example via the anti-free radical and anti-inflammatory effect of vitamin C.

The composition of the invention may comprise from 0.01 to 10% by weight of said at least one vitamin with respect to the total weight of the composition. For example, the composition may comprise from 0.01 to 5%, from 0.01 to 3%, from 0.01 to 2%, from 0.05 to 1%, or from 0.05 to 0.5% by weight of vitamin with respect to the total weight of the composition.

As used herein, “cosmetic composition” means any composition intended for cosmetic, that is aesthetic, use that can be brought into contact with the surface parts of the human body, for example the epidermis, hair and capillary systems, external organs, nails and external mucous membranes. Advantageously, a cosmetic composition can be used exclusively or principally to protect them, maintain them in good condition, modify their appearance or correct superficial defects. A cosmetic composition may comprise a cosmetically acceptable carrier.

As used herein, “dermatological composition” means any composition intended for dermatological use, that is a composition that can be brought into contact with the surface parts of the human body, for treatment of the skin, mucous membranes and appendages, for example nails, hair and/or body hair. A dermatological composition may comprise a dermatologically acceptable carrier.

The dermatological or cosmetic composition according to the invention may comprise one or more dermatologically and/or cosmetically acceptable carriers,

As used herein, “dermatologically and/or cosmetically acceptable carrier” means any cosmetic carrier known to the skilled person, for example any cosmetic carrier that can be cited in the INCI (International Nomenclature of Cosmetic Ingredients) dictionary published by the PCPC (Personal Care Products Council); it may, for example, be a carrier suitable for use in contact with human skin cells, in particular epidermal cells, without undue toxicity, irritation, allergic response and the like, and proportioned to a reasonable benefit/risk ratio. The cosmetically and/or dermatologically acceptable carrier may be selected from water, vaseline, beeswax, carnauba wax, stearic acid, cetostearyl alcohol, cetyl alcohol, diethylene glycol, isohexadecane, lanolin, glycerol monostearate, propylene glycol monostearate, cetyl palmitate, this list being non-limiting.

As used herein, “cosmetically acceptable carrier” means any cosmetic carrier known to the person skilled in the art, it may for example be any cosmetic carrier that can be cited in the INCI (International Nomenclature of Cosmetic Ingredients) dictionary published by the PCPC (Personal Care Products Council); it may for example be a carrier suitable for use in contact with human skin cells, in particular epidermal cells, without undue toxicity, irritation, allergic response and the like, and proportioned to a reasonable benefit/risk ratio. The cosmetically acceptable carrier may be selected from water, vaseline, beeswax, carnauba wax, stearic acid, cetostearyl alcohol, cetyl alcohol, diethylene glycol, isohexadecane, lanolin, glycerol monostearate, propylene glycol monostearate, cetyl palmitate, this list being non-limiting.

Advantageously, the cosmetically and/or dermatologically acceptable carrier may be vaseline.

The dermatological or cosmetic composition according to the invention may further comprise at least one solvent. It may be any solvent known to the skilled person suitable for use in a dermatological or cosmetic composition. It may for example be compounds or mixtures of compounds which dissolve, and which are capable of destroying the aggregation of molecules in a soluble body. It may for example be primary alcohols, for example ethanol, ethyl alcohol or isopropanol, or polyols, for example ethylene glycol, propylene glycol, triethylene glycol, tripropylene glycol, polyethylene glycol (PEG) or sorbitol. Advantageously, the solvent may be selected from the group comprising ethanol, propylene glycol or combinations thereof, preferably the solvent may be ethanol.

The composition of the invention may comprise from 0.5 to 5% by weight of solvent, for example from 1 to 5%, from 1 to 2% by weight of solvent, with respect to the total weight of the composition.

Advantageously, the solvent, preferably ethanol, can be used to solubilize said at least one vitamin in the composition according to the invention.

The dermatological or cosmetic composition according to the invention may comprise one or more adjuvants known to the person skilled in the art. Examples may include one or more adjuvants selected from ester-type agents, hydrating agents, emollients, mineral thickening agents, organic thickening agents, whether associative or not, water- and fat-soluble organic sunscreens, mineral sunscreens, silicone compounds, fragrances, preservatives, ceramides, and pseudoceramides, vitamins and provitamins, proteins, sequestering agents, alkalizing agents, acidifying agents, reducing agents, oxidizing agents, mineral fillers, colorants, or any other adjuvant listed in the INCI (International Nomenclature of Cosmetic Ingredients) dictionary published by the PCPC (Personal Care Products Council),

The dermatological or cosmetic composition according to the invention may further comprise one or more additional compound(s) selected from the group comprising at least one amino acid, polypeptide, desquamation agent, cell stimulant, firming agent, tensor, anti-aging agent, free radical scavenger and sun blocker, mixtures or combination thereof.

In the present context, amino acid means any amino acid known to the skilled person suitable for cosmetic use. It may for example be an amino acid from the Lor D series, preferably from the L series. Examples include citrulline, histidine, methionine, serine, tyrosine, cystine, cysteine and/or any mixture thereof.

As used herein, polypeptide means any polypeptide known to the skilled person suitable for cosmetic use. It may for example be tripeptide-1, palmitoyl tripeptide-1, myristoyl hexapeptide-16, myristoyl pentapeptide-17, hexanoyl dipeptide-3, Rh-polypeptide, Sh-polypeptide and/or any mixture thereof.

As used herein, desquamation agent means any desquamation agent known to the skilled person suitable for cosmetic or dermatological use. It may for example be AHA (α-hydroxy acids), papain, bromelain, stratum corneum chymotryptic enzyme (SCCE) and/or any mixture thereof.

As used herein, cellular stimulant means any cellular stimulant known to the skilled person and suitable for cosmetic or dermatological use. It may for example be any cellular stimulant acting on fibroblasts and their synthesis capacity known to the skilled person. It may for example be MSH (Melanocyte Stimulating Hormone), leukotrienes and/or any mixture thereof.

As used herein, firming agent means any firming agent known to the skilled person suitable for cosmetic or dermatological use. For example, any firming agent likely to improve the mechanical properties of the skin. It may for example be Crithmum maritimum.

As used herein, tensor means any tensor known to the skilled person suitable for cosmetic or dermatological use. It may for example be any tensor likely to reduce or eliminate superficial wrinkles and/or fine lines on the skin. It may for example be arginine and serine.

As used herein, anti-aging compound means any anti-aging compound known to the skilled person suitable for cosmetic or dermatological use. It may for example be any anti-aging compound of the estrogenic type which binds to epidermal receptors known to the skilled person. It may for example be 17-BEstradiol, benzoate estradiol, valerate estradiol and/or a mixture thereof.

As used herein, anti-free radical compound means any anti-free radical compound known to the skilled person suitable for cosmetic or dermatological use. It may for example be pre-tocopheryl glucoside, Vitamin C, Vitamin E and/or any mixture thereof.

As used herein, sunscreen compound means any sunscreen compound known to the person skilled in the art suitable for cosmetic or dermatological use. It may for example be any sunscreen compound that protects the skin from actinic radiation and/or UV rays. It may for example be water-soluble or lipid-soluble organic sunscreens and mineral sunscreens. It may for example be a sunscreen selected from the group comprising ethylhexyl methoxycinnamate, octocrylene, bis-ethylhexyloxyphenol methoxyphenyl triazine, ethylhexyl salicylate, butyl methoxydibenzoylmethane, titanium dioxide, zinc oxide and/or any mixture thereof.

Herein, the cosmetic or dermatological composition may comprise from 0.2 to 10% by weight of an adjuvant and/or additional compound, for example from 0.2 to 5% by weight of an adjuvant and/or additional compound relative to the total weight of the composition.

Herein, the adjuvant and/or additional compound may be selected from the group comprising xanthan gum, allantoin, oleic acid, sorbitan trioleate, sorbitan monostearate, soy lecithin, sorbitan monolaurate, polyoxyethylene sorbitan trioleate, polyoxyethylene sorbitan monooleate, potassium oleate and/or any mixture thereof.

The cosmetic or dermatological composition of the invention can be obtained by any suitable method known to the skilled person for the manufacture of a cosmetic or dermatological composition. It may for example be a simple mixture. It may for example be a method comprising a step of incorporating an internal phase into an external phase by means of an emulsifier, for example a rotor-stator type turbine. It may also be a method using Phase Inversion Temperature (PIT), this method being conventionally used by the skilled person to obtain oil-in-water emulsions whose dispersed droplets are particularly fine, for example with a diameter of 0.1 to 1 μm.

The cosmetic or dermatological composition of the present invention can be found in any form suitable for a cosmetic or dermatological application. Advantageously, the composition is a composition for topical use.

It may for example be a composition in a form selected from the group comprising a gel, an ointment, a cream, an oil, a milk, a salve, a powder, a soaked pad, a solution, a serum, a balm, a butter, a lotion, a suspension, a soap and an emulsion. Advantageously, the cosmetic or dermatological composition is in the form of a gel, cream or ointment.

The cosmetic or dermatological composition of the present invention may, for example, be in a form selected from the group comprising an aqueous or hydroalcoholic gel, an aqueous or hydroalcoholic cream and an aqueous or hydroalcoholic lotion. These formulations, which can be used to implement the present invention, are known to formulators in the state of the art.

According to the invention, the cosmetic or dermatological composition of the present invention may be free of preservatives.

In the present context, “preservative” means any preservative known to the skilled person suitable for use in a cosmetic or dermatological composition. It may be any preservative listed in the INCI (International Nomenclature of Cosmetic Ingredients) dictionary published by the PCPC (Personal Care Products Council).

As used herein, the composition may be administered topically. Administration may also depend on the area and/or biological tissue to be treated.

According to the invention, the cosmetic or dermatological composition of the invention may, for example, be administered once or several times.

According to the invention, the composition may be further administered, for example, daily, twice daily and weekly or less. It may be administered, for example, once a day, twice a day, three times a day or less, for example once every two days, or weekly. Advantageously, the composition may be administered daily twice a day.

According to the invention and the mode of administration, the composition may be, for example, for a composition in the form of a gel, administered daily, twice daily and weekly or less. It may be for example administration once-daily, twice-daily or less.

According to the invention, the composition may, for example, be administered over a period of 1 day to 21 days, for example 3 to 15 days, for example 3 to 7 days. For example, the composition may be administered over a 15-day period, for example with a daily or twice-daily administration frequency.

In other words, the composition may be administered, for example, over a period of 1 day to 45 days, for example 1 to 28 days, for example 3 to 15 days, for example 3 to 7 days. For example, the composition may be administered over a period of at least 15 days, for example with a frequency of administration every day and twice a day.

According to the invention, the composition of the present invention may be administered topically by simple application to the skin or by an application accompanied by massaging the skin and/or the area to which the composition is applied.

Another object of the present invention is a non-therapeutic cosmetic care method, comprising the application to the skin or mucous membranes of a cosmetic composition comprising at least one substituted or unsubstituted polylysine dendrimer, at least one vitamin and at least one cosmetically acceptable carrier.

The substituted or unsubstituted polylysine dendrimer is as defined hereinbefore.

The vitamin is as defined hereinbefore.

The cosmetically acceptable carrier is as defined hereinbefore.

According to the invention, the mode and/or route of administration of the cosmetic composition of the invention may be as defined hereinbefore, preferably by topical application.

According to the invention, the frequency of administration of the cosmetic composition of the invention may be as defined hereinbefore, preferably daily and twice a day.

According to the invention, cosmetic care may be selected from hydration, improving the appearance and soothing the skin and/or its appendages and/or mucous membranes.

According to the invention, cosmetic care may be selected from hydration, improving the appearance, improving skin suppleness, reducing skin tightness, soothing the skin and/or its appendages and/or mucous membranes.

Advantageously, hydration can be linked in whole or in part to an improvement in the trophicity of the stratum corneum, an improvement in cohesion between the corneocytes responsible for fixation of water, advantageously by virtue of a large quantity of hydrophilic keratin.

Advantageously, the improvement in skin appearance may be linked in whole or in part to a reduction in fine lines and wrinkles and/or an increase and/or strengthening of skin thickness and/or elasticity.

Advantageously, soothing may be a reduction in pain and/or itching and/or redness and/or skin lesions.

Advantageously, the improvement in skin suppleness and/or elasticity may be linked in whole or in part to increased synthesis of collagen and/or elastin at the dermal-epidermal junction and in the dermis.

Another object of the present invention is the non-therapeutic cosmetic use of a cosmetic composition comprising at least one substituted or unsubstituted polylysine dendrimer, at least one vitamin and at least one cosmetically acceptable carrier.

The substituted or unsubstituted polylysine dendrimer is as defined hereinbefore.

The vitamin is as defined hereinbefore.

The cosmetically acceptable carrier is as defined hereinbefore.

According to the invention, the mode and/or route of administration of the cosmetic composition of the invention may be as defined hereinbefore, preferably by topical application.

Another object of the present invention is the topical non-therapeutic cosmetic use of a cosmetic composition comprising at least one substituted or unsubstituted polylysine dendrimer, at least one vitamin and at least one cosmetically acceptable carrier.

According to the invention, the frequency of administration of the cosmetic composition of the invention may be as defined hereinbefore, preferably daily and twice a day.

According to the invention, the non-therapeutic cosmetic use can be for improving hydration, improving appearance, soothing the skin, reducing the tightness of the skin and/or its appendages and/or mucous membranes.

Advantageously, the non-therapeutic cosmetic use of a cosmetic composition comprising at least one substituted or unsubstituted polylysine dendrimer, at least one vitamin and at least one cosmetically acceptable carrier, is a use for improving the hydration of the skin and/or its appendages and/or mucous membranes.

Advantageously, hydration can be linked in whole or in part to an improvement in the trophicity of the stratum corneum, an improvement in cohesion between the corneocytes responsible for fixation of water, advantageously by virtue of a large quantity of hydrophilic keratin. Advantageously, hydration may also be linked to a reduction in transepidermal water loss by restoring the integrity of the stratum corneum and the correct function of the skin barrier.

Advantageously, the improvement in skin appearance may be linked in whole or in part to a reduction in fine lines and wrinkles and/or an increase and/or strengthening of skin thickness and/or elasticity.

Advantageously, soothing may be a reduction in pain and/or itching and/or redness.

Advantageously, the improvement in skin suppleness and/or elasticity may be linked in whole or in part to increased synthesis of collagen and/or elastin at the dermal-epidermal junction and in the dermis.

The inventor has also demonstrated in a surprising and unexpected way that the composition according to the invention advantageously allows a rapid reduction in lesions and/or irritation of the scalp, for example linked to actinic keratosis.

The inventor has also demonstrated in a surprising and unexpected way that the composition according to the invention advantageously allows a reduction up to the elimination of vulgar cutaneous warts or seborrhoeic palpebral warts.

The inventor has also demonstrated in a surprising and unexpected way that the composition according to the invention advantageously allows a reduction up to the elimination of cutaneous eczema.

The inventor has also demonstrated in a surprising and unexpected way that the composition according to the invention advantageously allows a reduction of cysts, for example Verneuil's disease. Surprisingly, the inventor has demonstrated that the composition according to the invention advantageously allows an attenuation of symptoms linked to inflammation, for example linked to edema and pain, the inventor has also demonstrated that the composition according to the invention advantageously allows a low cystic formation and advantageously allows a spontaneous evolution towards healing, advantageously without the use of any antibiotic treatment.

Another object of the present invention is a dermatological composition comprising at least one substituted or unsubstituted polylysine dendrimer, at least one vitamin and at least one dermatologically acceptable carrier for use in the treatment of a skin condition.

The substituted or unsubstituted polylysine dendrimer is as defined hereinbefore.

The vitamin is as defined hereinbefore.

The dermatologically acceptable carrier is as defined hereinbefore.

The inventor has surprisingly demonstrated that the composition according to the invention may be advantageously useful in the treatment of a skin condition involving skin inflammation and/or skin infection.

Another object of the present invention is a dermatological composition comprising at least one substituted or unsubstituted polylysine dendrimer, at least one vitamin and at least one dermatologically acceptable carrier for use in the treatment of a skin condition selected from the group comprising skin inflammation and/or a skin infection

The substituted or unsubstituted polylysine dendrimer is as defined hereinbefore.

The vitamin is as defined hereinbefore.

The dermatologically acceptable carrier is as defined hereinbefore.

According to the invention, the skin condition may be selected from the group comprising actinic keratosis, impetigo, pemphigus, dermatitis herpetiformis, atopic dermatitis, seborrheic dermatitis, allergic contact dermatitis, irritant contact dermatitis, lichen simplex chronicum, prurigo, pruritus, psoriasis, lichen planus, urticaria, sunburn, acute radiation dermatitis, hair loss, onycholysis, alopecia, acne, vitiligo, seborrheic keratosis, pressure sores, seborrheic warts, eczema, herpes, skin burns and Verneuil's disease.

According to the invention, the skin condition may be selected from the group comprising actinic keratosis, impetigo, pemphigus, dermatitis herpetiformis, atopic dermatitis, seborrheic dermatitis, allergic contact dermatitis, irritant contact dermatitis, lichen simplex chronicus, prurigo, pruritus, psoriasis, lichen planus, urticaria, sunburn, acute radiation dermatitis, hair loss, onycholysis, alopecia, vitiligo, seborrheic keratosis, pressure sores, seborrheic warts, eczema, herpes, skin burns. Examples include actinic keratosis, seborrheic keratosis, seborrheic dermatitis, psoriasis and acute radiation dermatitis.

According to the invention, the skin condition may be a condition comprising an allergic or immunoallergic inflammation and/or inflammatory reaction. It may for example be a skin condition selected from the group comprising pemphigus, atopic dermatitis, seborrheic dermatitis, allergic contact dermatitis, irritant contact dermatitis, lichen simplex chronicus, prurigo, pruritus, psoriasis, lichen planus, urticaria, alopecia, onycholysis, acne, vitiligo, seborrheic keratosis.

According to the invention, the skin condition may be a condition comprising an inflammation of physical or chemical radical origin, for example a burn, for example a chemical burn, acute radiation dermatitis, sunburn, a skin burn due to exposure to ultraviolet rays, for example UVA, UVB, preferably UVB, a pressure sore.

According to the invention, the skin condition may be a condition due to a microorganism, for example a bacterium or a virus. It may for example be a skin condition caused by a viral or bacterial infection. It may for example be a skin condition selected from the group comprising herpes, dermatitis herpetiformis and/or impetigo

Further advantages may become apparent to the skilled person upon reading the examples below, which are given by way of illustration.

### BRIEF DESCRIPTION OF THE FIGURES

FIG. 1 shows top-view photographs of the scalp before (FIG. 1A) or after (FIG. 1B) application of example compositions according to the invention. In the figures, the circle corresponds to the area of the scalp to which the composition was applied.

FIG. 2 shows top-view photographs of a human arm comprising a common wart before (FIG. 2A) and after 7 days (FIG. 2B) of application of an example composition according to the invention. On FIG. 2A, the circle corresponds to the area of the skin comprising a common wart.

FIG. 3 shows photographs of a human eyelid comprising a seborrheic wart before (FIG. 3A), after three days of application (FIG. 3B) or after 7 days of application (FIG. 3C) of an example composition according to the invention. In FIGS. 3A and 3B, the circle corresponds to the area of the skin comprising a seborrheic wart.

FIG. 4 shows top-view photographs of palms of hands showing eczema before (FIG. 4A), after three days of application (FIG. 4B) or after 7 days of application (FIG. 4C) of an example composition according to the invention.

FIG. 5 shows photographs of a groin showing a cyst before (FIG. 5A), after four days of application (FIG. 5B), after five days of application (FIG. 5C) of an example composition according to the invention and 21 days after application (FIG. 5D),

FIG. 6 shows photographs of hands with skin burns on the posterior surface of the hands before (FIGS. 6A-6B) and after three days of application (FIGS. 6C-6D) of an example composition according to the invention

FIG. 7 shows photographs of a mouth with herpes labialis before (FIG. 7A) and after three days of application (FIG. 7B) of an example composition according to the invention.

FIG. 8 shows a 3rd generation polylysine dendrimer of formula (VIII) FIG. 9 shows photographs of vials comprising a composition comprising at least one substituted or unsubstituted polylysine dendrimer, at least one vitamin and at least one cosmetically and/or dermatologically acceptable carrier. FIG. 9A and FIG. 9B show photographs of compositions after storage at a) 1 month at 25° C., b) 1 month at 4° C. and c) 90 hours at 45° C.

### EXAMPLES

### Example 1: Example Cosmetic and/or Dermatological Compositions

The cosmetic compositions were in the form of gels comprising vitamin A, also known as Retinol and derivatives and/or vitamin C, also known as ascorbic acid and derivatives. The poly-L-lysine dendrimers used were poly-L-lysine dendrimers in vaseline (Cera microcristallina/Paraffinum liquidum/Paraffin) marketed by Aiglon. In particular, the dendrimers were those marketed by Colcom under the trade name DGL G2-TFA and DGL G3-acetate. These were polylysine dendrimers obtained and as described in WO2006114528A1 or An Expeditious Multigram-Scale Synthesis of Lysine Dendrigraft (DGL) Polymers by Aqueous N-Carboxyanhydride Polycondensation. H. Collet and collaborators: Chem. Eur. J. 2010, 16, 2309-2316.

The vitamins used were vitamin C, also known as ascorbic acid or vitamin A also known as retinol acetate marketed respectively by Sigma-Aldrich, France.

Each composition was prepared from a base of approximately 10 g Vaseline, weighed into a PolyPropylene (PP) screw-top jar. An olive-shaped magnetic bar (approximately 2 cm) was added to each jar then heated to 55-65° C. until the Vaseline liquefied. To this liquid Vaseline was added, whilst stirring, an aqueous solution comprising a mixture of poly-L-lysine dendrimers and vitamins previously homogenized by ultrasound. The preparation was continuously stirred for 2 hours. The magnetic bar was removed from the still-liquid Vaseline, and the samples were stored in a refrigerator at 2-6° C.

Examples of the compositions obtained are described in Tables 1 and 2 below. In these tables, percentages are given by mass with respect to the total mass of the composition.

TABLE 1
Example composition
Example composition (1)

| | % by weight/% by weight of |
|---|---|
| INCI/Name Raw material (RM) | formula |
| Aqua (water) | 2 |
| Ascorbic acid | 0.5 |
| (Vaseline) | 97.48 |
| Cera microcristallina Paraffinum liquidum | |
| Paraffin | |
| DGL G2-TFA | 0.02 |

TABLE 2
Example composition
Example composition (2)

| | % by weight/% by weight of |
|---|---|
| INCI/Name Raw material (RM) | formula |
| Alcohol: ethanol | 1 |
| Retinyl acetate | 0.05 |
| (Vaseline) | 98.9 |
| Cera microcristallina Paraffinum liquidum | |
| Paraffin | |
| DGL G3-acetate | 0.05 |

### Example 2: Determination of Skin Tolerance of Example Compositions Comprising Poly-L-Lysine Dendrimers and at Least One Vitamin

In this example, the skin tolerance of cosmetic and/or dermatological compositions was determined by measuring the primary irritant potential of said compositions after a single 15-minute application in a volunteer.

This is a single-center, open-label trial in adult volunteers, with no direct individual benefit. The sample size required to test the tolerance of the compositions was 12 subjects in an open-label design. The main criteria for inclusion were as follows: volunteers, over 18 years of age, in good health, non-allergic, with skin free of any dermatological lesions, and having freely agreed to sign the information and consent form.

All 12 subjects received the same compositions. The composition was applied undiluted on the back to the shoulder blade then rinsed off with clear water after 15 minutes of contact under normal conditions of use, once only, to a skin site on the back of each volunteer.

The clinical evaluation consisted of a macroscopic skin examination, which was carried out immediately in daylight, 30 minutes and 24 hours after removal of the composition.

An average irritation score was determined: PII=total reaction score, total number of volunteers, reactions were rated from 0 to 4.

The parameters examined were as follows:
the morphological appearance of the skin by a dermatological examination, looking for erythema (redness), edema (swelling), papules (small solid red elevations, granular sensation to the touch) and vesicles (small detachments of the skin containing a drop of liquid), the subjective skin sensitivity by the volunteer according to the following five criteria graded from 0 to 3 (0=absent, 1=slight, 2=moderate, 3=severe),
pain,
burn,
tingling,
itching, and
any “other events”.

Skin reactions (erythema, edema, etc.) were assessed according to the criteria of the International Contact Dermatitis Research Group (ICDRG), presented as follows:
NT: Not tested?
?+: Questionable reaction. Slight erythema only.
+: Weak positive reaction (not vesicular): erythema, infiltration, sometimes a few papules.
++: Strong positive reaction: presence of erythema, papules, vesicles.
+++: Extremely positive reaction, with the presence of bubbles.
−: Negative reaction
IR: Irritant reaction: E0.5: very slight erythema
E1: mild erythema
E2: significant erythema
E3: severe erythema

The results were interpreted as follows:

The primary irritation index at each reading time was calculated according to the ratio:
PII=Σ of erythematous scores/Number of subjects.

The scale for interpreting skin irritation was as follows:
If PII<0.20 non-irritating
If 0.20≤PII<0.50 slightly irritating
If 0.50≤PII<1 moderately irritating
If PII≥1 irritating

If a reaction is observed on both the control and treated sites, the intensity of the product-induced reaction is given by the difference between the treated site and the control site.

An assessment of possible skin reactions and the intensity thereof, namely erythema, edema, infiltration, papule, vesicle, necrosis, was therefore carried out according to the protocol of the International Contact Dermatitis Research Group (ICDRG) as described in Fregert S. Manual of Contact Dermatitis. 2nd ed. Munksgaard, Copenhagen: Year Book Medical Publishers, Inc; 1981. Chapter 10, Patch Testing; pp. 71-81.

In this example, 16 compositions described in Table 3 below were tested and a comparison of said compositions was made based on their acute skin tolerance in single or multiple applications. The compositions tested were obtained according to the method described in Example 1 hereinbefore. In particular, the dendrimers used were respectively dendrimers marketed by Colcom under the trade name DGL G2-TFA, DGL G2-acetate, DGL G3-TFA or DGL G3-acetate. These were polylysine dendrimers obtained and as described in WO2006114528A1 or An Expeditious Multigram-Scale Synthesis of Lysine Dendrigraft (DGL) Polymers by Aqueous N-Carboxyanhydride Polycondensation. H. Collet and collaborators: Chem. Eur. J. 2010, 16, 2309-2316.

The compositions tested included Vitamin C, also known as ascorbic acid, marketed by Sigma-Aldrich laboratories, France.

Acute skin tolerance was assessed by a 15-minute patch test in 12 healthy volunteers aged at least 18 years in accordance with strict ethical requirements. Of these 12 volunteers, 5 had sensitive skin regardless of any treatment (5/12). Macroscopic examinations, comprising a dermatological clinical examination of the skin including an assessment of the parameters examined as mentioned hereinbefore, were carried out immediately, 30 minutes and 24 hours after removal of the product. Two volunteers applied these formulations daily for a week.

In Table 3, the percentages are given by mass with respect to the total mass of the composition.

TABLE 3
Compositions used

| | | | % by mass with |
|---|---|---|---|
| | | | respect to the |
| | | | total |
| | | CAS | mass of the |
| Compositions | INCI | Number | composition |
| Composition a | Aqua (water) | 7732-18-5 | 2 |
| | Ascorbic acid | 50-81-7 | 0.5 |
| | Cera microcristallina | 63231-60-7 | 97.48 |
| | Paraffinum liquidum | 8042-47-5 | |
| | Paraffin | 8002-74-2 | |
| | DGL G2-TFA | | 0.02 |
| Composition b | Aqua (water) | 7732-18-5 | 2 |
| | Ascorbic acid | 50-81-7 | 0.5 |
| | Cera microcristallina | 63231-60-7 | 97.45 |
| | Paraffinum liquidum | 8042-47-5 | |
| | Paraffin | 8002-74-2 | |
| | DGL G2-TFA | | 0.05 |
| Composition c | Aqua (water) | 7732-18-5 | 2 |
| | Ascorbic acid | 50-81-7 | 0.5 |
| | Cera microcristallina | 63231-60-7 | 97.48 |
| | Paraffinum liquidum | 8042-47-5 | |
| | Paraffin | 8002-74-2 | |
| | DGL G3-TFA | | 0.02 |
| Composition d | Aqua (water) | 7732-18-5 | 2 |
| | Ascorbic acid | 50-81-7 | 0.5 |
| | Cera microcristallina | 63231-60-7 | 97.45 |
| | Paraffinum liquidum | 8042-47-5 | |
| | Paraffin | 8002-74-2 | |
| | DGL G3-TFA | | 0.05 |
| Composition e | Alcohol | 64-17-5 | 1 |
| | Retinyl acetate | 127-47-9 | 0.05 |
| | Cera microcristallina | 63231-60-7 | 98.93 |
| | Paraffinum liquidum | 8042-47-5 | |
| | Paraffin | 8002-74-2 | |
| | DGL G3-acetate | | 0.02 |
| Composition f | Alcohol | 64-17-5 | 1 |
| | Retinyl acetate | 127-47-9 | 0.05 |
| | Cera microcristallina | 63231-60-7 | 98.9 |
| | Paraffinum liquidum | 8042-47-5 | |
| | Paraffin | 8002-74-2 | |
| | DGL G3-acetate | | 0.05 |
| Composition g | Alcohol | 64-17-5 | 1 |
| | Retinyl acetate | 127-47-9 | 0.05 |
| | Cera microcristallina | 63231-60-7 | 98.93 |
| | Paraffinum liquidum | 8042-47-5 | |
| | Paraffin | 8002-74-2 | |
| | DGL G3-acetate | | 0.02 |
| Composition h | Alcohol | 64-17-5 | 1 |
| | Retinyl acetate | 127-47-9 | 0.05 |
| | Cera microcristallina | 63231-60-7 | 98.9 |
| | Paraffinum liquidum | 8042-47-5 | |
| | Paraffin | 8002-74-2 | |
| | DGL G3-TFA | | 0.05 |
| Composition 1 | Aqua (water) | 7732-18-5 | 2 |
| | Ascorbic acid | 50-81-7 | 0.5 |
| | Cera microcristallina | 63231-60-7 | 97.48 |
| | Paraffinum liquidum | 8042-47-5 | |
| | Paraffin | 8002-74-2 | |
| | DGL G2-TFA | | 0.02 |
| Composition j | Aqua (water) | 7732-18-5 | 2 |
| | Ascorbic acid | 50-81-7 | 0.5 |
| | Cera microcristallina | 63231-60-7 | 97.45 |
| | Paraffinum liquidum | 8042-47-5 | |
| | Paraffin | 8002-74-2 | |
| | DGL G2-TFA | | 0.05 |
| Composition k | Aqua (water) | 7732-18-5 | 2 |
| | Ascorbic acid | 50-81-7 | 0.5 |
| | Cera microcristallina | 63231-60-7 | 97.48 |
| | Paraffinum liquidum | 8042-47-5 | |
| | Paraffin | 8002-74-2 | |
| | DGL G3-TFA | | 0.02 |
| Composition 1 | Aqua (water) | 7732-18-5 | 2 |
| | Ascorbic acid | 50-81-7 | 0.5 |
| | Cera microcristallina | 63231-60-7 | 97.45 |
| | Paraffinum liquidum | 8042-47-5 | |
| | Paraffin | 8002-74-2 | |
| | DGL G3-TFA | | 0.05 |
| Composition m | Alcohol | 64-17-5 | 1 |
| | Retinyl acetate | 127-47-9 | 0.05 |
| | Cera microcristallina | 63231-60-7 | 98.93 |
| | Paraffinum liquidum | 8042-47-5 | |
| | Paraffin | 8002-74-2 | |
| | DGL G3-acetate | | 0.02 |
| Composition o | Alcohol | 64-17-5 | 1 |
| | Retinyl acetate | 127-47-9 | 0.05 |
| | Cera microcristallina | 63231-60-7 | 98.9 |
| | Paraffinum liquidum | 8042-47-5 | |
| | Paraffin | 8002-74-2 | |
| | DGL G3-acetate | | 0.05 |
| Composition p | Alcohol | 64-17-5 | 1 |
| | Retinyl acetate | 127-47-9 | 0.05 |
| | Cera microcristallina | 63231-60-7 | 98.93 |
| | Paraffinum liquidum | 8042-47-5 | |
| | Paraffin | 8002-74-2 | |
| | DGL G3-acetate | | 0.02 |
| Composition q | Alcohol | 64-17-5 | 1 |
| | Retinyl acetate | 127-47-9 | 0.05 |
| | Cera microcristallina | 63231-60-7 | 98.9 |
| | Paraffinum liquidum | 8042-47-5 | |
| | Paraffin | 8002-74-2 | |
| | DGL G3-TFA | | 0.05 |

The results obtained were determined from the twelve volunteers included in the study. Tables 4 and 5 below show the different results obtained based on the compositions and subjects. In Tables 4 and 5, the treated site corresponds to the application to the skin of the shoulder blade of a patch, also referred to as a pad, comprising an example composition according to the invention, and the control site corresponds to the contralateral application to the skin of the shoulder blade of a patch, also referred to as a pad, comprising no cosmetic or dermatological composition.

All volunteers completed the study without any deviation from the protocol.

TABLE 4
Results of the skin reaction/tolerance assessment

| | | | | CONTROL SITE | |
|---|---|---|---|---|---|
| | | TREATED SITE | | Observation | Observation |
| | | Observation | Observation | 30 minutes | 24 hours |
| | | 30 minutes | 24 hours | after patch | after patch |
| | | after patch | after patch | removal | removal |
| SUBJECTS | | removal | removal | Negative | Negative |
| | Age | Composition | Composition | control | control |
| Identification | (gender) | (reaction) | (reaction) | (reaction) | (reaction) |
| SY.R | 51(F) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| MY.B | 20(F) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| SA. B | 18(F) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| HA. B | 59(M) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| IL.H | 34(F) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| SA. H | 37(F) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| DJ.B | 45(M) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| AN.F | 33(F) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| HE.R | 48(F) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| EM.T | 28(M) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| CH.H | 30(F) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |
| LI.B | 26(F) | a(—), b(—), c(—), | a(—), b(—), c(—), | (—) | (—) |
| | | d(—), e(—), f(—), | d(—), e(—), f(—), | | |
| | | g(—), h(—), i(—), | g(—), h(—), i(—), | | |
| | | j(—), k(—), l(—), | j(—), k(—), l(—), | | |
| | | m(—), o(—), p(—), | m(—), o(—), p(—), | | |
| | | q(—) | q(—) | | |

TABLE 5
Results of the assessment of the primary irritation index (PII)

| | | Treated site | | Control site | |
|---|---|---|---|---|---|
| | | 30 minutes | 24 hours | 30 minutes | 24 hours |
| | | after patch | after patch | after patch | after patch |
| Compositions | | removal | removal | removal | removal |
| a | PII | 0 | 0 | 0 | 0 |
| | AVERAGE | | | | |
| | Results | Non- | Non- | Non- | Non- |
| | | irritating | irritating | irritating | irritating |
| b | PII | 0 | 0 | 0 | 0 |
| | AVERAGE | | | | |
| | Results | Non- | Non- | Non- | Non- |
| | | irritating | irritating | irritating | irritating |
| C | PII | 0 | 0 | 0 | 0 |
| | AVERAGE | | | | |
| | Results | Non- | Non- | Non- | Non- |
| | | irritating | irritating | irritating | irritating |
| d | PII | 0 | 0 | 0 | 0 |
| | AVERAGE | | | | |
| | Results | Non- | Non- | Non- | Non- |
| | | irritating | irritating | irritating | irritating |
| e | PII | 0 | 0 | 0 | 0 |
| | AVERAGE | | | | |
| | Results | Non- | Non- | Non- | Non- |
| | | irritating | irritating | irritating | irritating |
| f | PII | 0 | 0 | 0 | 0 |
| | AVERAGE | | | | |
| | Results | Non- | Non- | Non- | Non- |
| | | irritating | irritating | irritating | irritating |
| g | PII | 0 | 0 | 0 | 0 |
| | AVERAGE | | | | |
| | Results | Non- | Non- | Non- | Non- |
| | | irritating | irritating | irritating | irritating |
| h | PII | 0 | 0 | 0 | 0 |
| | AVERAGE | | | | |
| | Results | Non- | Non- | Non- | Non- |
| | | irritating | irritating | irritating | irritating |
| i | PII | 0 | 0 | 0 | 0 |
| | AVERAGE | | | | |
| | Results | Non- | Non- | Non- | Non- |
| | | irritating | irritating | irritating | irritating |
| j | PII | 0 | 0 | 0 | 0 |
| | AVERAGE | | | | |
| | Results | Non- | Non- | Non- | Non- |
| | | irritating | irritating | irritating | irritating |
| k | PI | 0 | 0 | 0 | 0 |
| | AVERAGE | | | | |
| | Results | Non- | Non- | Non- | Non- |
| | | irritating | irritating | irritating | irritating |
| l | PII | 0 | 0 | 0 | 0 |
| | AVERAGE | | | | |
| | Results | Non- | Non- | Non- | Non- |
| | | irritating | irritating | irritating | irritating |
| m | PII | 0 | 0 | 0 | 0 |
| | AVERAGE | | | | |
| | Results | Non- | Non- | Non- | Non- |
| | | irritating | irritating | irritating | irritating |
| o | PII | 0 | 0 | 0 | 0 |
| | AVERAGE | | | | |
| | Results | Non- | Non- | Non- | Non- |
| | | irritating | irritating | irritating | irritating |
| P | PII | 0 | 0 | 0 | 0 |
| | AVERAGE | | | | |
| | Results | Non- | Non- | Non- | Non- |
| | | irritating | irritating | irritating | irritating |
| q | PII | 0 | 0 | 0 | 0 |
| | AVERAGE | | | | |
| | Results | Non- | Non- | Non- | Non- |
| | | irritating | irritating | irritating | irritating |

In Table 5 hereinbefore, the PII AVERAGE corresponds to the average of the PII values measured in the 12 subjects.

As shown in Tables 4 and 5 above, the results obtained demonstrated an absence of erythema, edema, papules, vesicles and/or necrosis at the skin surface. As demonstrated by the results of macroscopic examinations, all the compositions tested showed very good skin tolerance, both in volunteers with normal skin and in volunteers with sensitive skin. In addition, no adverse events and/or side effects were observed.

This example therefore clearly demonstrates that example compositions according to the invention are compositions with high skin tolerance, causing no possible side effects. This example also clearly demonstrates that example compositions according to the invention do not induce inflammatory or allergic reactions.

### Example 3: Effect of an Example Composition Comprising Poly-L-Lysine Dendrimers and at Least One Vitamin on Actinic Keratosis of the Scalp

In this example, the compositions used were compositions 1 and 2 as described in Example 1 hereinbefore. Applications of composition to a scalp lesion of the actinic keratosis type were carried out by applying 1 cm³ (1 mL) of composition to the scalp, extending one centimeter beyond the area to be treated, lightly massaging using a circular inward-outward movement, for 15 days, applying composition 1 in the morning and composition 2 in the evening. Before each application, the skin was rinsed with soapy water.

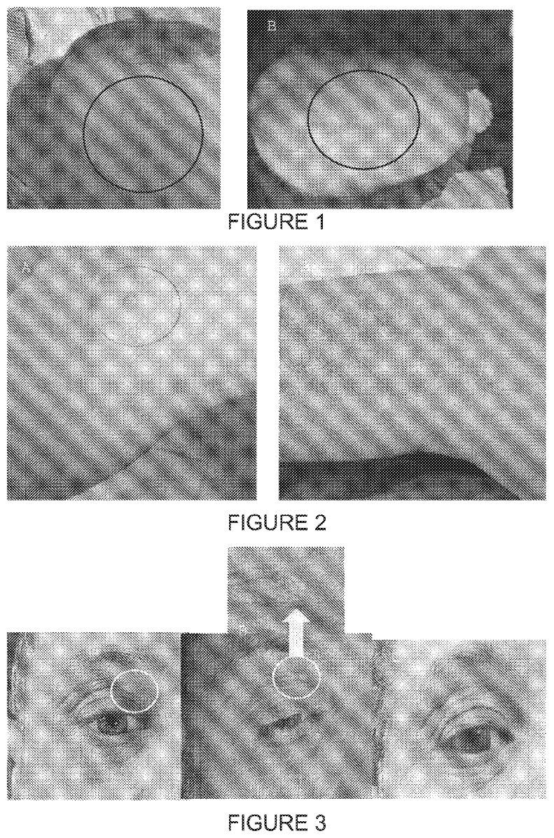

FIG. 1 shows photographs of the scalp before application of compositions 1 and 2 (FIG. 1A) and after 15 days of application with compositions 1 and 2.

From the very first application of the compositions, pruritus and the desire to scratch were reduced. These results clearly demonstrate the rapid effect of the composition, particularly in the treatment of pruritus of the scalp. Additionally, as shown in FIG. 1B, after 15 days of application, the appearance of the scalp was normal, that is, it no longer comprised scabs, lesions and scarring.

The results clearly demonstrate that example compositions according to the invention are useful for improving the appearance of the skin, in particular of the scalp. The results also clearly demonstrate that example compositions according to the invention advantageously allow treatment of skin conditions, for example actinic keratosis. The results also clearly demonstrate that example compositions according to the invention advantageously allow treatment of skin lesions induced by ultra-violet rays and/or exposure to the sun.

### Example 4: Effect of an Example Composition Comprising Poly-L-Lysine Dendrimers and at Least One Vitamin after Application to a Common Skin Wart

In this example, the compositions used were compositions 1 and 2 as described in Example 1 hereinbefore. Applications of the composition to a common wart were carried out by applying 1 cm³ (1 mL) of composition to the skin, extending one centimeter beyond the area to be treated, that is, including the common wart, lightly massaging using a circular inward-outward movement, for 15 days, using composition 1 in the morning and composition 2 in the evening. Before each application, the skin was rinsed with soapy water.

FIG. 2 shows photographs of the antero-external surface of the right arm before application of compositions 1 and 2 (FIG. 2A) and after 7 days of application of compositions 1 and 2.

As shown in FIG. 2B, after 7 days of application, the common wart was absent, and the appearance of the skin was normal. Additionally, no recurrence was observed after treatment.

The results clearly demonstrate that example compositions according to the invention are useful for improving skin appearance. Additionally, this example clearly demonstrates that example compositions according to the invention advantageously allow treatment of skin conditions, for example a skin lesion of the verruca vulgaris type (B07 Viral warts) which is included in the list of Viral Infections characterized by skin and mucosal lesions according to the International Classification of Diseases (B07, ICD10, Chapter I),

### Example 5: Effect of an Example Composition Comprising Poly-L-Lysine Dendrimers and at Least One Vitamin after Application to a Seborrheic Palpebral Wart

In this example, the compositions used were compositions 1 and 2 as described in Example 1 hereinbefore. Applications of a composition to a seborrheic wart located on an eyelid were carried out by applying 1 cm³ (1 mL) of composition to the skin, extending one centimeter beyond the area to be treated, that is, including the seborrheic wart, lightly massaging using a circular inward-outward movement, for 7 days, using composition 1 in the morning and composition 2 in the evening. Before each application, the skin was rinsed with soapy water.

FIG. 3 shows photographs of the right eyelid before application of compositions 1 and 2 (FIG. 3A), after 3 days (FIG. 3B) and after 7 days (FIG. 3C) of application of compositions 1 and 2.

As shown in FIG. 3C, after 7 days of application, the seborrheic wart was absent, and the appearance of the skin was normal. Additionally, no recurrence was observed after treatment. The results clearly demonstrate that example compositions according to the invention are useful for improving skin appearance. Additionally, this example clearly demonstrates that example compositions according to the invention advantageously allow treatment of skin conditions, for example a skin lesion of the seborrheic wart type ((L82) Seborrhoeic Keratosis) which forms part of the list of Diseases of the skin and subcutaneous cellular tissue according to the International Classification of Diseases (L82, ICD10, Chapter XII),

### Example 6: Effect of an Example Composition Comprising Poly-L-Lysine Dendrimers and at Least One Vitamin after Application to Skin Eczema

In this example, the compositions used were compositions 1 and 2 as described in Example 1 hereinbefore. Applications of compositions to eczema located on the palms of hands were carried out by applying 2 cm³ (2 mL) of composition to the skin, extending one centimeter beyond the area to be treated, that is, including the eczematous skin lesion, lightly massaging using a circular inward-outward movement, for 7 days, using composition 1 in the morning and composition 2 in the evening. Before each application, the skin was rinsed with soapy water.

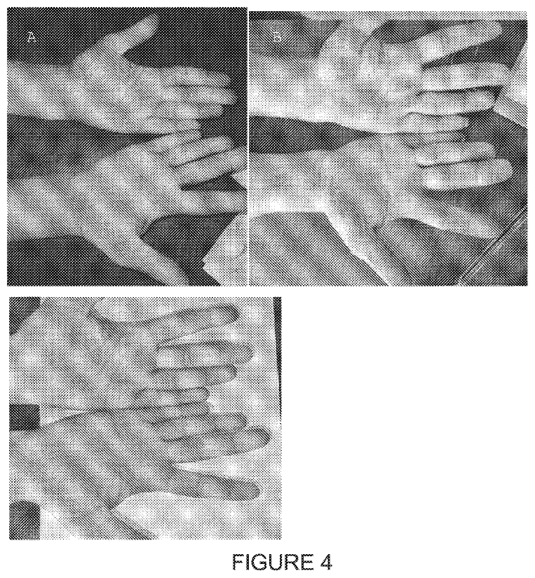

FIG. 4 shows photographs of the palms of the hands before application of compositions 1 and 2 (FIG. 4A), after 3 days (FIG. 4B) and after 7 days (FIG. 4C) of application of compositions 1 and 2.

The results showed a disappearance of itching affecting the skin lesions after the first application, followed by desquamation on the third day and disappearance of these lesions on the seventh day.

As shown in FIG. 4C, after 7 days of application, skin appearance was normal.

The results clearly demonstrate that example compositions according to the invention are useful for improving skin appearance. Additionally, this example clearly demonstrates that example compositions according to the invention advantageously allow treatment of skin conditions, for example a skin lesion of the eczema type ((L20-L30) Dermatitis and eczema (syndrome)) which is part of the list of Diseases of the skin and subcutaneous cellular tissue according to the International Classification of Diseases (L20-L30, ICD10, Chapter XII),

### Example 7: Effect of an Example Composition Comprising Poly-L-Lysine Dendrimers and at Least One Vitamin after Application to a Verneuil's Disease Cyst

In this example, the compositions used were compositions 1 and 2 as described in Example 1 hereinbefore. Applications of the composition to a cystic flare-up located at an inguinal groove were carried out by applying 1 cm³ (1 mL) of composition to the skin, extending one centimeter beyond the area to be treated, that is, including the cyst, lightly massaging using a circular inward-outward movement, for 5 days, using composition 1 in the morning and composition 2 in the evening. Before each application, the skin was rinsed with soapy water.

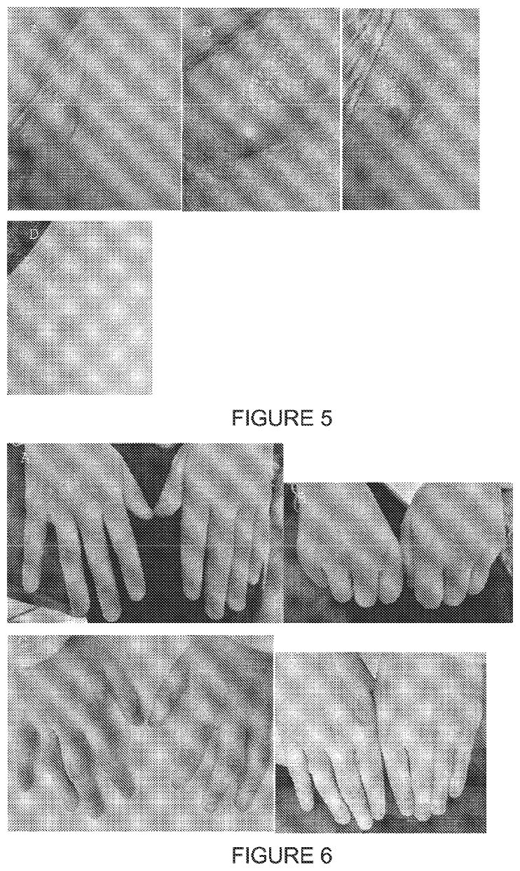

FIG. 5 shows photographs of the groin before application of compositions 1 and 2 (FIG. 5A), after 4 days (FIG. 5B), after 5 days (FIG. 5C) of application of compositions 1 and 2, and 21 days after application (FIG. 5D),

As shown in FIG. 5B and observed, by the third day, a move towards a circumscribed inflammatory lesion was observed. The inflammatory lesion was subsequently easily removed and healed without complication and in the absence of antibiotics.

The results clearly demonstrate that example compositions according to the invention are useful for improving skin appearance. Additionally, this example clearly demonstrates that example compositions according to the invention are useful in the treatment of skin conditions, for example a skin lesion of the Verneuil's disease type ((L73.2) Hidradenitis suppurativa) which is part of the list of Diseases of the skin and subcutaneous cellular tissue according to the International Classification of Diseases (L73.2, ICD10, Chapter XII),

### Example 8: Effect of an Example Composition Comprising Poly-L-Lysine Dendrimers and at Least One Vitamin after Application to a Skin Burn

In this example, the compositions used were compositions 1 and 2 as described in Example 1 hereinbefore. Applications of compositions to a skin burn on the posterior surface of the hands following caustic soda spraying were carried out by applying 3 cm³ (3 mL) of composition to the skin, extending one centimeter beyond the area to be treated, that is, including the burn area, lightly massaging using a circular inward-outward movement, for 3 days, using composition 1 in the morning and composition 2 in the evening. Before each application, the skin was rinsed with soapy water.

FIG. 6 shows photographs of the hands before application of compositions 1 and 2 (FIGS. 6A and 6B), and after 3 days (FIGS. 6C and 6D) of application of compositions 1 and 2.

From the very first application of the composition, disappearance of pain was observed. As shown in FIGS. 6C and 6D, on the third day of application, skin appearance was normal, with no lesions.

The results clearly demonstrate that example compositions according to the invention are useful for improving skin appearance. Additionally, this example clearly demonstrates that example compositions according to the invention are useful in the treatment of skin burns, for example a caustic chemical burn injury ((T23) Burn and corrosion of wrist and hand) which is part of the list of Burns and Corrosions according to the International Classification of Diseases (T23, ICD10, Chapter XIX),

### Example 9: Effect of an Example Composition Comprising Poly-L-Lysine Dendrimers and at Least One Vitamin after Application to Herpes Labialis

In this example, the compositions used were compositions 1 and 2 as described in Example 1 hereinbefore. Applications of compositions to a herpes labialis were carried out by applying 1 cm³ (1 mL) of composition to the skin, extending one centimeter beyond the area to be treated, that is including the herpes lesion, lightly massaging using a circular inward-outward movement, for 3 days, using composition 1 in the morning and composition 2 in the evening. Before each application, the skin was rinsed with soapy water.

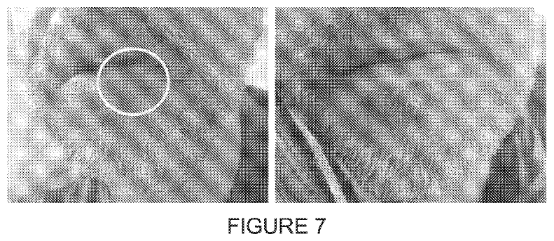

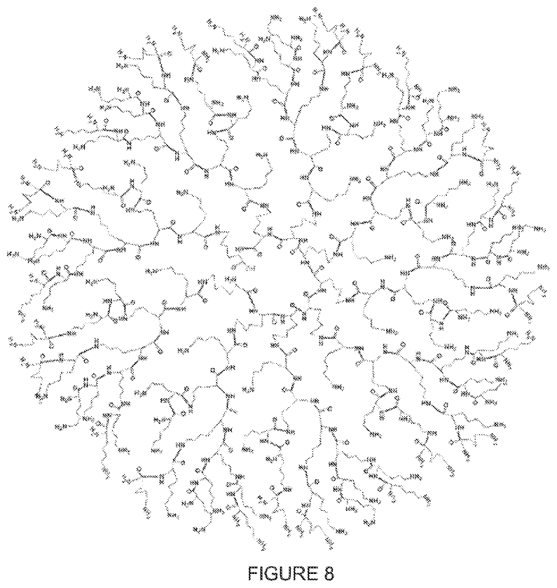

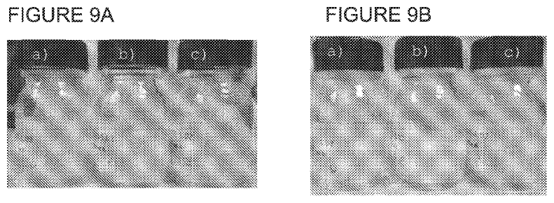

FIG. 7 shows photographs of the lips before application of compositions 1 and 2 (FIG. 7A), and after 3 days (FIG. 7B) of application of compositions 1 and 2.

From the very first application of the composition, disappearance of pain was observed. As shown in FIG. 7B, on the third day of application, skin appearance was normal, with no lesions.

The results clearly demonstrate that example compositions according to the invention are useful for improving skin appearance. Furthermore, this example clearly demonstrates that examples of compositions according to the invention are useful in the treatment of a skin lesion of the herpes type ((L13.0) Dermatitis herpetiformis) which is included in the list of Diseases of the skin and subcutaneous cellular tissue according to the International Classification of Diseases (L13.0, ICD10, Chapter XII),

### Example 10: Example Cosmetic and/or Dermatological Compositions

The compositions were in the form of a gel comprising vitamin A, also known as Retinol and derivatives and/or vitamin C, also known as ascorbic acid and derivatives. The poly-L-lysine dendrimers used were DGL G2-TFA and DGL G3-acetate poly-L-lysine dendrimers marketed by COLCOM. These were polylysine dendrimers obtained according to the method described in documents WO2006114528A1 or An Expeditious Multigram-Scale Synthesis of Lysine Dendrigraft (DGL) Polymers by Aqueous N-Carboxyanhydride Polycondensation. H. Collet and collaborators: Chem. Eur. J. 2010, 16, 2309-2316 [2]

The vitamins used were vitamin C, also known as ascorbic acid or vitamin A also known as retinol acetate marketed respectively by Sigma-Aldrich, France.

Each composition was prepared according to the method comprising the following steps: in a beaker containing a mixture of water, glycerin and sodium polyacrylate, mechanically stirred at 600 rpm with a shredding disk and heated to 70°±5° C., to which a hot mixture (70°±2° C.) of sweet almond oil, isopropyl palmitate and tropolone was added. The solution was continuously stirred at 3000 rpm and at 70°±2° C. for 15 minutes. The solution was then brought to a temperature of 40°±5° C. while stirring at 600 rpm, then the fragrance and a mixture of poly-L-lysine dendrimers and vitamins previously homogenized by ultrasound were added. The resulting emulsion was continuously stirred at 600 rpm at 40°±5° C. for 15 minutes. Samples were stored in a refrigerator at 2-6° C.

Example compositions obtained are described in Tables 6 and 7 below. In these tables, percentages are given by mass with respect to the total mass of the composition.

TABLE 6
Example composition comprising polylysine dendrimers, particularly
of the G3 generation, and vitamin A (Composition I)

| | | % by mass with |
|---|---|---|
| | | respect to the total |
| | | mass of the |
| Name (INCI) Mixed | CAS | composition |
| AQUA (WATER) | 7732-18-5 | 84.6 |
| ISOPROPYL PALMITATE | 142-91-6 | 5.0 |
| PRUNUS AMYGDALUS DULCIS | 8007-69-0 | 4.0 |
| (SWEET ALMOND) OIL | | |
| GLYCERIN | 56-81-5 | 3.0 |
| SODIUM POLYACRYLATE | 25549-84-2 | 11.3 |
| DENATURED ALCOHOL | 64-17-5 | 1 |
| FRAGRANCE | / | 0.5 |
| POLYLYSINE (DGL G3-acetate) | 25104-18-1 | 0.05 |
| RETINOL | 68-26-8 | 0.05 |
| TROPOLONE | 533-75-5 | 0.5 |
| Total | | 100.0 |

TABLE 7
Example composition comprising polylysine dendrimers, particularly
of the G2 generation, and vitamin C (Composition H)

| Name (INCI) Mixed | CAS | % INCI |
|---|---|---|
| AQUA (WATER) | 7732-18-5 | 85.38 |
| ISOPROPYL PALMITATE | 142-91-6 | 5.0 |
| PRUNUS AMYGDALUS DULCIS (SWEET | 8007-69-0 | 4.0 |
| ALMOND) OIL | | |
| GLYCERIN | 56-81-5 | 3.0 |
| SODIUM POLYACRYLATE | 25549-84-2 | 1.6 |
| ASCORBIC ACID | 50-81-7 | 0.5 |
| PARFUM (FRAGRANCE) | / | 0.5 |
| POLYLYSINE (DGL G2-TFA) | 25104-18-1 | 0.02 |
| Total | | 100.0 |

### Example 11: Stability Testing of Cosmetic and/or Dermatological Compositions

In this example, the stability of the compositions 5 described in Tables 6 (Composition I) and 7 (Composition II) was assessed.

The conditions under which the assessment was carried out are described in Table 8 below:

TABLE 8
Protocol for assessing composition stability

| Test conditions | | Test description | Duration |
|---|---|---|---|
| Actual | Control at | This product is the control, it | 3 months |
| conditions | 25° C. and in | serves as a reference. It is | |
| | the dark | stored at 25° C. away from light | |
| Aging | Study at | Determination of average shelf | 3 months |
| conditions | 45° C. and in | life. 3 months at 45° C. are | |
| | the dark | equivalent to 3 years of normal | |
| | | storage | |
| Extreme | Refrigerator | temperature conditions: 4° C. | 1 month |
| conditions | at 4° C. and in | Product stability under extreme | |
| | the dark | | |
| | UV light | Assessment of a possible | 90 hours |
| | booth (45° C.) | change in the exposed product, | |
| | | storage at 45° C. UV lamp | |
| | | 300 W, 12 hours/day, 5 out of | |
| | | 7 days. | |

The results are shown in Tables 9 and 10.

The composition is considered compliant if the appearance, color and odor remain unchanged, and the measured parameters, especially pH and viscosity, do not deviate by more than 20% from their original values (Knowlton, John L., and Steven EM Pearce. Handbook of Cosmetic Science & Technology. Elsevier, 2013 [11].

The appearance of the composition was assessed by a daylight inspection, looking for significant changes in color, fluidity and texture, with the presence of flocculation, sedimentation and creaming according to the procedure described in the European Cosmetics Association. “Cosmetics Europe: Guidelines on stability testing of cosmetic products. Guidel Stab Test Cosmet Prod [Internet], Cosmetics Europe; 2004 March” and Romanowski, Perry and Randy Schueller. “Stability testing of cosmetic products.” Novel Cosmetic Delivery Systems 6 (2023): 115-129 [12].

TABLE 9
Stability results for composition 1

| | STABILITY | | | | |
|---|---|---|---|---|---|
| | | Control | | | |
| Duration | | 24 h | 15 days | 1 month | 3 months |
| Oven | — | | Very very | Slight | Yellowing and |
| 45° C. | | | slight | yellowing | reduced fragrance |
| | | | yellowing | | |
| 4° C. | — | | Compliant | Compliant | Keep cold like |
| | | | | | control |
| Control | APPEARANCE | Cream gel | Compliant | Very slight | Slight yellowing |
| 25° C. | COLOR | No color, | | yellowing | |
| | | white | | | |
| | ODOR | White | | | |
| | | musk | | | |
| | | 2005957 | | | |
| | | at 0.5% | | | |
| | pH (25° C., | 5.5 +/− 0.5 | | | 5.74 |
| | 24 hours) | | | | |

| UV/45° C. | — | Very slight yellowing and slight change in |
|---|---|---|
| (90 hours) | | odor |

FIG. 9A shows composition 1 after a) 1 month at 25° C., b) 1 month at 4° C. and c) 90 hours at 45° C.

The results obtained show that the accelerated aging test (3 months at 45° C.) of composition 1 shows stability of the 5 organoleptic characteristics (appearance, odor, color), that is, no significant degradation of the composition.

Additionally, the results show that under normal conditions of use and storage (storage at room temperature: 15-25° C., protected from light, heat, humidity and under hermetic conditions), the estimated shelf life of composition 1 is greater than 30 months.

TABLE 10
Stability results for composition 2

| | STABILITY | | | | |
|---|---|---|---|---|---|
| Duration | | 24 hour control | 15 days | 1 month | 3 months |
| Oven 45° C. | | | Compliant | Compliant | Compliant |
| 4° C. | | | Compliant | Compliant | |
| 25° C. | APPEARANCE | Cream gel | Compliant | Compliant | Compliant |
| control | COLOR | No color, white | | | |
| | ODOR | White musk | | | |
| | | 2005957 at | | | |
| | | 0.5% | | | |
| | pH (25° C., | 5.5 +/− 0.5 | — | — | 5.45 |
| | 24 hours) | | | | |

| | |
|---|---|
| UV/45° C. | Very slight yellowing and slight |
| (90 hours) | change in odor |

FIG. 9B shows composition 2 after a) 1 month at 25° C., b) 1 month at 4° C. and c) 90 hours at 45° C.

The results obtained demonstrate that the accelerated aging test (3 months at 45° C.) of composition 2 shows stability of organoleptic characteristics (appearance, odor, color), that is, no significant degradation of the composition

Additionally, the results show that under normal conditions of use and storage (storage at room temperature: 15-25° C., protected from light, heat, humidity and under hermetic conditions), the estimated shelf life of composition 2 is greater than 30 months.

### Example 11: Hydrating Effect of Cosmetic or Dermatological Compositions after a Single Application

In this example, an assessment of skin hydration by the compositions described in Tables 6 (Composition 1) and 7 (Composition 2) was carried out by applying the composition to the skin of the leg (treated site), with the control site (untreated) being located on the contralateral leg and not comprising any application of compositions.

11 adult female volunteers, aged between 40 and 70, average age 65, with dry skin on the legs (corneometric values less than or equal to 50 A.U.) were included in the study.

In this study, 0.07 mL of pure product, namely composition 1 or 2, was applied to a 35 cm² area of skin on the leg and efficacy was assessed by corneometric measurements after three hours. The measurements were carried out by the same person throughout the study.

The hydrating effect was measured by determining the degree of hydration of the upper layers of the epidermis by measuring the electrical capacity of the stratum corneum. A very low-intensity electric field was generated on the surface of the skin by the Corneometer probe. Electrical capacitance was then measured, proportional to the degree of hydration of the stratum corneum. Electrical capacitance measurements were carried out using a Corneometer® CM 825 probe (Courage+Khazaka Electronic GmbH, Cologne, Germany), on a 49 mm² surface area. The electrical capacitance values of the stratum corneum were given in arbitrary units, on a scale from 0 to 130 A.U.

For each volunteer and each measurement time, only the mean of the 3 repeat measurements was taken into account. This average value forms the basis of the individual data.

From the individual data, the following descriptive statistics are given for each measurement time:
Mean, standard deviation;
Percentage changes:
Gross percentage change (without taking into account changes in the control area), calculated as follows:

$$gross \% change = ( PTX - PTO ) / PTO$$

where: PTX=average of values at TX over the treated area PT0=average of values at T0 over the treated area
% relative change, taking into account changes recorded over the control area, calculated as follows:

$$\% relative change = ( ( ( PTX - PTO ) \times 100 ) / PTO ) - ( ( ( CTX - CTO ) \times 100 ) / CTO ) )$$

where: PTX=average of values at TX over the treated area PT0=average of values at T0 over the treated area CTX=average of values at TX over the control area CTO=average of values at T0 over the control area

Table 11 shows the averages and standard deviations of hydration for the sites (skin parts) to which composition 1 and the control were applied and the results of the statistical analysis.

TABLE 11
Hydration averages and standard deviations

| Time | | T0 | T1 h | T2 h | T3 h |
|---|---|---|---|---|---|
| Composition 1 | Average | 24.86 | 36.50 | 42.03 | 43.68 |
| site | (A.U.) | | | | |
| | Standard | 3.23 | 7.04 | 7.06 | 7.08 |
| | deviation | | | | |
| Control site | Average | 27.85 | 29.47 | 28.72 | 30.49 |
| | (A.U.) | | | | |
| | Standard | 6.33 | 8.06 | 7.71 | 7.05 |
| | deviation | | | | |

| | | | | |
|---|---|---|---|---|
| Paired Wilcoxon test | — | p = | P = | p = |

| | | |
|---|---|---|
| 0.003 | 0.003 | 0.003 |
| (S) | (S) | (S) |

In Table 11, S stands for significant difference (p≤0.05), NS stands for non-significant difference (p>0.05) and A.U. stands for arbitrary units: values measured by the corneometer.

Table 12 shows the percentage change in the degree of hydration after application of composition 1.

TABLE 12
Percentage change in degree of hydration

| Time after application of composition | T1 h | T2 h | T3 h |
|---|---|---|---|
| % change in treated site | 46.8% | 69.0% | 75.7% |
| % change in control site | 5.8% | 3.1% | 9.5% |
| % change in treated site - control site | 41.0% | 65.9% | 66.2% |

As shown, the statistical analysis using the Wilcoxon test for paired data between Tx and TO for composition 1 revealed a statistically significant 41.0% increase in the degree of hydration at T1h, a statistically significant 65.9% increase in the degree of hydration at T2h, and a statistically significant 66.2% increase in the degree of hydration at T3h.

Table 13 shows the averages and standard deviations for hydration of the sites (skin parts) to which composition 2 and the control were applied, and the results of the statistical analysis.

TABLE 13
Hydration averages and standard deviations

| Time | | T0 | T1 h | T2 h | T3 h |
|---|---|---|---|---|---|
| Composition 2 | Average | 25.62 | 36.90 | 42.18 | 41.37 |
| site | (A.U.) | | | | |
| | Standard | 5.44 | 10.66 | 5.89 | 9.88 |
| | deviation | | | | |
| Control site | Average | 27.85 | 29.47 | 28.72 | 30.49 |
| | (A.U.) | | | | |
| | Standard | 6.33 | 8.06 | 7.71 | 7.05 |
| | deviation | | | | |

| | | | | |
|---|---|---|---|---|
| Paired Wilcoxon test | — | p = 0.003 | p = 0.003 | p = 0.004 |

| | | |
|---|---|---|
| (S) | (S) | (S) |

In Table 13, S stands for significant difference (p≤0.05), NS stands for non-significant difference (p>0.05) and A.U. for arbitrary units: values measured by the corneometer.

Table 14 shows the percentage change in the degree of hydration after application of composition 2.

TABLE 14
Percentage change in degree of hydration

| Time after application of composition | T1 h | T2 h | T3 h |
|---|---|---|---|
| % change in site after application of | 44.0% | 64.6% | 61.5% |
| the composition | | | |
| % change in control site | 5.8% | 3.1% | 9.5% |
| % change in site with composition - | 38.2% | 61.5% | 52.0% |
| control site | | | |

As demonstrated, the statistical analysis using the Wilcoxon test for paired data, between Tx and TO, for composition 2 showed a statistically significant increase in the degree of hydration of 38.2% at T1h, a statistically significant increase in the degree of hydration of 61.5% at T2h, and a statistically significant increase in the degree of hydration of 52% at T3h.

The results therefore demonstrate that the use of compositions comprising a substituted or unsubstituted polylysine dendrimer, at least one vitamin, for example vitamin A or vitamin C, and at least one cosmetically and/or dermatologically acceptable carrier advantageously allows hydration of the skin, particularly of the superficial layers of the epidermis, in a statistically significant way at each measurement time.

This example therefore clearly demonstrates that example compositions according to the invention or the use of a composition comprising a substituted or unsubstituted polylysine dendrimer, at least one vitamin, for example vitamin A or vitamin C, and at least one cosmetically and/or dermatologically acceptable carrier can significantly increase skin hydration in a very short time. This example also demonstrates that the use of a composition comprising a substituted or unsubstituted polylysine dendrimer, at least one vitamin, for example vitamin A or vitamin C, and at least one cosmetically and/or dermatologically acceptable carrier can significantly increase the hydration of dry skin.

### Example 12: Evaluation of Clinical and Dermatological Tolerance and Efficacy after Use and Use Test

In this example, an evaluation of the clinical and dermatological tolerance and acceptability of example compositions comprising a substituted or unsubstituted polylysine dendrimer, at least one vitamin, for example vitamin

A or vitamin C, and at least one cosmetically and/or dermatologically acceptable carrier, namely composition 1 and 2 described hereinbefore, was carried out.

The composition was applied 2 times a day to clean, dry skin on the face and hands, and once a day to dry areas of the body (forearms, elbows and legs) over a 28-day period. A skin assessment was carried out before application of the composition and 28 days after application of the composition.

Composition 1 included 22 volunteers comprising 19 women and 3 men, with an average age of 63, ranging from 34 to 70 years. All volunteers had dry to very dry facial skin and 11 volunteers reported sensitive facial skin.

Composition 2 included 22 volunteers comprising 21 women and 1 man, with an average age of 63, ranging from 47 to 70 years. All the volunteers had dry to very dry facial skin and 11 reported sensitive facial skin.

Clinical and dermatological tolerance was assessed by clinical examination through inspection and skin palpation by an expert dermatologist. The examinations were carried out under environmental conditions appropriate to the study and were performed by the same assessor (investigator or clinical assistant under the investigator's responsibility) throughout the study. Each criterion was graded from 0 to 3 (0=absent; 0.5= very mild; 1=mild; 2=moderate; 3=severe), with no clinical signs of intolerance or discomfort.

An evaluation of example compositions comprising at least one substituted or unsubstituted polylysine dendrimer, at least one vitamin, for example vitamin A or vitamin C, and at least one cosmetically and/or dermatologically acceptable carrier, namely composition 1 and 2 described hereinbefore, on the efficacy of treating damaged skin, namely, rough, cracked skin responsible for discomfort, tightness and itching, and on the nourishing effect of said compositions, in particular by assessing the dryness and suppleness of the skin was carried out. The evaluation was carried out by comparing the skin before application of the composition and after 28 days of application of the composition. Evaluation of skin condition: damaged or undamaged, dryness and suppleness was carried out according to a score from 0 to 10, namely, damaged skin: 0=skin not damaged; 10=skin extremely damaged; skin dryness: 0=skin not dry; 10=skin extremely dry; skin suppleness: 0=skin not supple; 10=skin extremely supple. The evaluation was carried out by clinical examination by an expert dermatologist through inspection and skin palpation.

Tables 15 and 16 show the averages, standard deviations, minimum and maximum values and percentages of variation for damaged and undamaged skin, skin dryness and suppleness, and the results of the statistical analysis after application of composition 1 or 2 respectively.

TABLE 15
Results concerning the condition, dryness and
suppleness of the skin with composition 1

| | | | Nourishing effect | | | |
|---|---|---|---|---|---|---|
| | Damaged skin | | Skin dryness | | Skin suppleness | |
| | D1 | D28 | D1 | D28 | D1 | D28 |
| Average | 7.2 | 3.7 | 7.7 | 4.3 | 3.3 | 6.3 |
| Standard | 0.9 | 0.8 | 1.1 | 1.6 | 0.9 | 1.1 |
| deviation | | | | | | |
| Minimum | 6 | 2 | 6 | 2 | 2 | 4 |
| Maximum | 19 | 5 | 9 | 7 | 5 | 8 |
| Percentage | | −48.4 | | −43.8 | | 89.0 |
| change (%) | | | | | | |
| p-value | | p < 0.000 | | p < 0.000 | | p < 0.000 |
| Significance | | (S) | | (S) | | (S) |

TABLE 16
Results concerning the condition, dryness and
suppleness of the skin with composition 2

| | | | Nourishing effect | | | |
|---|---|---|---|---|---|---|
| | Damaged skin | | Skin dryness | | Skin suppleness | |
| | D1 | D28 | D1 | D28 | D1 | D28 |
| Average | 5.8 | 3.3 | 7.1 | 4.5 | 2.9 | 5.9 |
| Standard | 0.9 | 0.9 | 1.6 | 1.3 | 1.3 | 1.1 |
| deviation | | | | | | |
| Minimum | 4 | 2 | 4 | 2 | 1 | 4 |
| Maximum | 7 | 5 | 10 | 6 | 5 | 8 |
| Percentage | | −43.8 | | −37.2 | | 100.0 |
| change (%) | | | | | | |
| p-value | | p < 0.000 | | p < 0.000 | | p < 0.000 |
| Significance | | (S) | | (S) | | (S) |

In Tables 15 and 16, the statistical test is the Wilcoxon test for paired data, S stands for Significant (p≤0.05),

As demonstrated, composition 1 or 2 significantly improves skin condition, suppleness and hydration over time.

An example evaluation of compositions comprising at least one substituted or unsubstituted polylysine dendrimer, at least one vitamin, for example vitamin A or vitamin C, and at least one cosmetically and/or dermatologically acceptable carrier, namely compositions 1 and 2 described hereinbefore, on skin tightness was carried out. The evaluation was carried out by volunteers by comparing the skin before application of the composition and after 28 days of application of the composition. The evaluation of tightness was carried out using a score from 0 to 10, namely: 0=no tightness; 10=considerable tightness, by questioning subjects using a visual analog scale.

Tables 17 and 18 show the averages, standard deviations, minimum and maximum values and percentage change in skin tightness after application of composition 1 or 2 respectively.

TABLE 17
Results concerning skin tightness with composition 1

Tightness

| | D 1 | D 28 |
|---|---|---|
| Average | 7.3 | 2.8 |
| Standard deviation | 1.2 | 1.2 |
| Minimum | 5 | 0 |
| Maximum | 9 | 5 |
| Percentage change (%) | | −62.1 |
| p-value Significance | | p < 0.000 |
| | | (S) |

TABLE 18
Results concerning skin tightness with composition 2

Tightness

| | D 1 | D 28 |
|---|---|---|
| Average | 6.5 | 4.2 |
| Standard deviation | 1.5 | 1.4 |
| Minimum | 5 | 2 |
| Maximum | 10 | 7 |
| Percentage change (%) | | −34.5 |
| p-value Significance | | p < 0.000 |
| | | (S) |

In Tables 17 and 18, the statistical test is the Wilcoxon test for paired data, S stands for Significant (p≤0.05),

As demonstrated, applying a composition comprising a substituted or unsubstituted polylysine dendrimer, at least one vitamin, for example vitamin A or vitamin C, and at least one cosmetically and/or dermatologically acceptable carrier to the skin results in a significant reduction in tightness.

An evaluation of cosmetic acceptability by volunteers was also carried out after using composition 1 or 2 for 28 days. The results showed an overall satisfaction of 95% and 91% respectively, with over 75% of volunteers also validating the following properties of the composition:
The scent is pleasant
The texture is pleasant
The product is absorbed rapidly
The product is easy to apply
The product does not leave the skin sticky
The skin is soft
The skin is supple
The product improves the overall appearance of the skin
The product provides a feeling of freshness
The product leaves a protective film on the skin
The product provides protection against external aggressors
The product respects the skin
The skin is intensely nourished
The skin regains its comfort
The product hydrates the skin all day long
The product reduces tightness
The product suits your skin type

As demonstrated, compositions comprising a substituted or unsubstituted polylysine dendrimer, at least one vitamin, for example vitamin A or vitamin C, and at least one cosmetically and/or dermatologically acceptable carrier have excellent cosmetic and dermatological tolerance on dry to very dry skin, particularly on the face. Furthermore, compositions comprising at least one substituted or unsubstituted polylysine dendrimer, at least one vitamin, for example vitamin A or vitamin C, and at least one cosmetically and/or dermatologically acceptable carrier did not induce any feeling of discomfort or reaction in volunteers who reported having sensitive skin, thus clearly demonstrating that these compositions are useful for non-therapeutic cosmetic use and/or care and/or treatment of sensitive skin, for example sensitive facial skin. This example also clearly demonstrates that compositions comprising at least one substituted or unsubstituted polylysine dendrimer, at least one vitamin, for example vitamin A or vitamin C, and at least one cosmetically and/or dermatologically acceptable carrier have a skin-nourishing effect, for example advantageously helping to reduce dryness and increase skin suppleness. This example also clearly demonstrates that compositions comprising at least one substituted or unsubstituted polylysine dendrimer, at least one vitamin, for example vitamin A or vitamin C, and at least one cosmetically and/or dermatologically acceptable carrier have a skin repairing effect, an anti-tightness effect and very good cosmetic acceptability.

### Example 13: Evaluation of the Skin Compatibility of Compositions Comprising a Substituted or Unsubstituted Polylysine Dendrimer, at Least One Vitamin, for Example Vitamin A or Vitamin C, and at Least One Cosmetically and/or Dermatologically Acceptable Carrier

An evaluation of skin compatibility of compositions comprising at least one substituted or unsubstituted polylysine dendrimer, at least one vitamin, for example vitamin A or vitamin C, and at least one cosmetically and/or dermatologically acceptable carrier was carried out by patch testing on the anterolateral side of the arm for 48 hours.

An occlusive dressing (Finn Chamber® or equivalent) comprising 20 μL (or 0.02 g) of pure composition was applied to the anterolateral side of the arm for 48 hours.

Skin compatibility of the composition was assessed 48 hours after the initial application, between 30 and 40 minutes after removal of the dressing. Skin reactions (erythema and edema) were scored from 0 to 3 using the scales described in Table 19 below.

TABLE 19
Skin reaction score

| Score | Erythema (E) | Edema (OE) |
|---|---|---|
| 0 | no erythema | no edema |
| 0.5 | barely perceptible erythema, | palpable, barely perceptible edema |
| | very slightly pinkish coloring | |
| | over part of the patch area | |
| 1 | mild erythema, pinkish | palpable and visible edema |
| | coloring over the entire | |
| | patch surface area | |
| 2 | moderate erythema, clear | obvious edema with or without |
| | coloring over the entire | papules or vesicles |
| | patch surface area | |
| 3 | severe erythema, intense | severe edema spreading outside the |
| | coloring over the entire | patch area, with or without papules |
| | patch area | or vesicles |

Any other skin reactions (bullae, papules, vesicles, dryness, desquamation, roughness, soap effect, etc.) were also assessed according to the following scale and reported descriptively: 0: no reaction, 0.5: very slight, 1: slight, 2: moderate, and 3: significant.

A primary irritation index (PII) was calculated using the following formula:

$$1.1 . M . = ( Sum of skin reactions ( E + OE + bullae + papules + vesicles ) ) / Number of volunteers tested$$

The PII obtained was used to classify the composition tested according to the scale shown in Table 20.

TABLE 20
Classification of composition according to the PII

| | |
|---|---|
| PII ≤ 0.20 | Non-irritating |
| 0.20 < PII ≤ 0.50 | Slightly irritating |
| 0.50 < PII ≤ 2 | Moderately irritating |
| 2 < PII ≤ 3 | Severely irritating |

For composition 1, the number of volunteers included was 11 individuals. The PII result obtained was 0 and no side effects were identified.

For composition 2, the number of volunteers included was 11 individuals. The PII result obtained was 0 and no side effects were identified.

A test to assess the ocular irritation potential of composition 1 or 2 by studying cytotoxicity after agarose gel diffusion on the NCTC L929 cell line was also carried out. This test is an in vitro method for assessing ocular irritation potential. The principle is based on determining the cytotoxicity of the test element, namely the composition, 24 hours after application of the composition to the surface of an agarose gel in contact with a cell monolayer. Cell viability was assessed by mitochondrial succinate dehydrogenase activity in living cells. This enzyme converts MTT into blue formazan crystals. The range of cell lysis revealed by staining is linked to the diffusion capacity of the test element through the agarose gel. The measurement of the diameter of the lysis range is proportional to the cytotoxicity of the test element. This test was carried out according to the protocol published in the Official Journal of the French Republic (No. 302) on Dec. 30, 1999. The cells used were NCTC L929 mouse lung fibroblasts (ATCC-CCLI); the cells were mycoplasma-free. The media used were:
complete culture medium: DMEM 1 g/L glucose, 2 mM L-glutamine or stabilized glutamine, 10% stripped FBS, 50 IU/mL penicillin, 50 μg/mL streptomycin, 1% non-essential amino acids-stored at 5° C.±3° C.
Complete medium concentrated 2 times: DMEM 2X, 4 mM L-glutamine or stabilized glutamine, 20% stripped FBS, 100 IU/mL penicillin, 100 μg/mL streptomycin—prepared extemporaneously and used on the same day
Dulbecco's PBS without CA²⁺ and Mg²⁺-stored at room temperature-Trypsin EDTA IX (0.5 g/l porcine trypsin, 0.2 g/l EDTA)—stored at
20° C.±5° C.
2% agarose solution in ultrapure water-stored at 8° C.±3° C.
Staining solution: MTT (3-(4,5-dimethylthiazol-2-yl)-2,5-diphenyltetrazolium bromide) solution at 0.5 mg/mL in DMEM 1 g/L (not supplemented)—prepared extemporaneously and used on the same day

The positive control was a 3% solution of SDS (CAS number: 151-21-3) and the negative control of the complete culture medium.

The test was carried out as follows:

Cells were trypsinized and counted. Petri dishes were seeded with 4.5 mL of a cell suspension at 2.5×10 cells/mL in complete culture medium, then cultured overnight (37° C., 5% CO₂). At the end of incubation, cell layer confluence was checked. The 2% agarose gel was melted by heating in a water bath at around 80° C. and mixed volume by volume with 2× concentrated complete medium. After removing the culture medium by aspiration, 4.5 mL of 1% agarose complete medium (temperature around 37° C.) was poured into each dish. After being left to solidify for a few minutes at room temperature, the dishes were placed at 37° C., 5% CO₂ for 30±10 minutes to allow stabilization of the reagent system. A disk of filter paper (6 mm diameter) was placed in the center of each dish on the surface of the agarose gel. Then, 10 μL of composition 1 or 2 (test element) and reference elements (positive control or negative control) were carefully deposited on the disks.

The test system was exposed to composition 1 or 2 and to the reference elements for 24 hours±1 hour at 37° C., 5% CO₂.

At the end of the treatment time, the agarose gel was carefully removed using a spatula without damaging the cell layer. Development was carried out at 37° C., 5% CO₂ by adding 2 mL of staining solution to each dish for 1 hour±30 minutes. The solution was then removed by inversion or suction. After cell staining, an unstained area (lysis zone) may appear in the center of the dish. To account for diffusion heterogeneity, the largest and smallest diameters of this zone were measured with a sheet of graph paper and the mean diameter (MD) was calculated. The cytotoxicity of the composition corresponds to the arithmetic mean of the mean diameters measured on the four dishes. This value is expressed in centimeters to two decimal places

The results obtained clearly demonstrated the absence of cytotoxicity of the compositions and an absence of eye irritation and/or eye irritation potential.

A test to assess a comedogenic effect of compositions comprising at least one substituted or unsubstituted polylysine dendrimer, at least one vitamin, for example vitamin A or vitamin C, and at least one cosmetically and/or dermatologically acceptable carrier, in particular composition 1 or 2, was carried out.

The composition was applied once a day to clean facial skin over a 28-day period. A skin assessment was carried out before application of the composition and 28 days after application of the composition. A clinical dermatological examination was carried out by an expert dermatologist, including an inspection in daylight and palpation of the facial skin. Scoring consisted of counting all skin lesions, namely, comedones, microcysts, papules and pustules that may be present.

For composition 1, the number of volunteers included was 21, with an average age of 35, ranging from 19 to 45 years. All the volunteers had oily skin with a tendency to acne and imperfections, and 11 volunteers said they had sensitive facial skin.

For composition 2, the number of volunteers included was 24, with an average age of 36, ranging from 20 to 45 years. All the volunteers had oily skin on their faces, with a tendency to acne and imperfections, and 12 volunteers said they had sensitive facial skin.

Daily application of formulations 1 and 2 consisted of applying a hazelnut-sized amount (around 2 mL) of one composition to the entire face, lightly massaging using a circular inward-outward movement.

All volunteers underwent a clinical examination (site examined: face) with the acne lesions counted. Table 21 shows the corresponding results.

TABLE 21
Characteristics of volunteers

| | | | Comedones | | |
|---|---|---|---|---|---|
| | | | and | | |
| | Number | Age | microcysts | Papules | Pustules |
| Composition 1 | 21 | Mean: 35 | 21 (100%) | N = 3 | N = 0 |
| | | Between: | | (9.5%) | (0%) |
| | | 19-45 years | | | |
| Composition 2 | 24 | Average: 36 | 24 (100%) | N = 7 | N = 0 |
| | | Between: | | (29.2%) | (0%) |
| | | 20-45 years | | | |

Tables 22 and 23 show the means, standard deviations, minimum and maximum values and percentage relating to the presence of comedones and microcysts, papules, pustules or nodules and cysts, and the result of the statistical analysis after application of composition 1 or 2 respectively.

TABLE 22
Results concerning the presence of comedones and microcysts,
papules, pustules or nodules and cysts after application of composition 1

| | | | comedones and | | | | | | | |
|---|---|---|---|---|---|---|---|---|---|---|
| | Total score | | microcysts | | papules | | pustules | | nodules and cysts | |
| | D1 | D28 | D1 | D28 | D1 | D28 | D1 | D28 | D1 | D28 |
| Average | 12.5 | 12.9 | 12.1 | 12.0 | 0.4 | 5.9 | 0.0 | 0.0 | 0.0 | 0.0 |
| Standard | 2.3 | 2.6 | 2.3 | 2.1 | 1.0 | 1.4 | 0.0 | 0.0 | 0.0 | 0.0 |
| deviation | | | | | | | | | | |
| Minimum | 9 | 9 | 9 | 9 | 0 | 0 | 0 | 0 | 0 | 0 |
| Maximum | 18 | 20 | 18 | 18 | 4 | 4 | 0 | 0 | 0 | 0 |
| Percentage | — | 3.1 | — | −0.8 | — | NA | — | NA | | NA |
| change (%) | | | | | | | | | | |
| p-value | — | P < 0.000 | — | P < 0.000 | — | | — | | | |
| Significance | | (S) | | (S) | | | | | | |

TABLE 23
Results concerning the presence of comedones and microcysts,
papules, pustules or nodules and cysts after application of composition 2

| | Total | | comedones and | | | | | | nodules | |
|---|---|---|---|---|---|---|---|---|---|---|
| | score | | microcysts | | papules | | pustules | | and cysts | |
| | D1 | D28 | D1 | D28 | D1 | D28 | D1 | D28 | D1 | D28 |
| Average | 127 | 10.8 | 10.9 | 9.6 | 1.9 | 1.0 | 0.0 | 0.0 | 0.0 | 0.0 |
| Standard | 6.1 | 2.7 | 3.0 | 1.7 | 4.1 | 2.4 | 0.0 | 0.0 | 0.0 | 0.0 |
| deviation | | | | | | | | | | |
| Minimum | 7 | 6 | 7 | 6 | 0 | 0 | 0 | 0 | 0 | 0 |
| Maximum | 36 | 16 | 20 | 14 | 16 | 8 | 0 | 0 | 0 | 0 |
| Percentage | — | −15.1 | — | −12.2 | — | NA | — | NA | — | NA |
| change (%) | | | | | | | | | | |

In Tables 22 and 23, the statistical test is the Wilcoxon test for paired data, NS means Not Significant (p>0.05), NA means Not Applicable.

As shown in Tables 22 and 23 hereinbefore, the number of comedones and microcysts, papules, pustules or nodules and cysts did not increase after 28 days of application.

The results in Tables 22 and 23 show a negative change in the “comedones and microcyst count” score of −0.8% or −12.2%, clearly demonstrating a reduction in the number of comedones and microcysts after a 28-day application of composition 1 or 2.

As demonstrated, compositions comprising a substituted or unsubstituted polylysine dendrimer, at least one vitamin, for example vitamin A or vitamin C, and at least one cosmetically and/or dermatologically acceptable carrier have excellent cosmetic and dermatological tolerance on oily or combination oily skin, particularly on the face.

As demonstrated, compositions comprising a substituted or unsubstituted polylysine dendrimer, at least one vitamin, for example vitamin A or vitamin C, and at least one cosmetically and/or dermatologically acceptable carrier are devoid of comedogenic potential.

Additionally, the sebum-regulating effect of the compositions tested was also studied. To do this, an expert dermatologist observed and rated the “oily appearance” of the skin: 0.5: very slight; 1: slight; 2: moderate; 3: significant. For composition 1, the number of volunteers included was 21, with an average age of 35, ranging from 19 to 45 years. All the volunteers had oily facial skin.

In particular, a comparative test comprising the application of a sufficient quantity of the composition corresponding to a hazelnut, about 2 mL, to the entire face, lightly massaging using a circular inward-outward movement, of composition 1 or 2.

Table 24 summarizes the results obtained.

TABLE 24
Skin oiliness

| | |
|---|---|
| Composition 1 One | Composition 2 One |
| daily application | daily application |

| Formulation | D 1 | D 28 | D 1 | D 28 |
|---|---|---|---|---|
| Total score | 33 | 27 | 37 | 35 |
| Average | 1.57 | 1.28 | 1.54 | 1.46 |

| | | |
|---|---|---|
| % change | −18.47 | −5.20% |

As shown in Table 24, the use of compositions comprising a substituted or unsubstituted polylysine dendrimer, at least one vitamin, for example vitamin A or vitamin C, and at least one cosmetically and/or dermatologically acceptable carrier advantageously helps to reduce the oily appearance of the skin.

Additionally, a study of the anti-imperfection effect, namely comedones, microcysts, papules and pustules, of the compositions tested was also carried out. In particular, an improvement/anti-imperfection effect corresponded to a decrease in the total imperfection score: number of comedones, microcysts, papules and pustules, and the absence of effect corresponded to no decrease or increase in the total imperfection score. For this purpose, a dermatological expert observed the skin, scoring skin imperfections at the start (D1) and at the end of the study (D28). All volunteers had facial imperfections: (i) 21 subjects aged between 19 and 45 years applied composition 1 to their facial skin on a daily basis, massaging lightly using a circular inward-outward movement, and (ii) 24 subjects aged between 20 and 35 applied composition 2 to their facial skin on a daily basis, massaging lightly using a circular inward-outward movement.

Table 25 summarizes the results obtained.

TABLE 25
Results concerning the anti-imperfection effect

| | | | Total |
|---|---|---|---|
| | Improvement | No effect | number |
| Composition 1 | N = 5 (24%) | N = 16 (76%) | N = 21 |
| | | | (100%) |
| Composition 2 | N = 18 (75%) | N = 6 (25%) | N = 24 |
| | | | (100%) |

| | |
|---|---|
| Significance* | Highly significant difference (α = 0.001) |

As shown in Table 25, the results obtained show an improvement in the appearance of the skin after daily application of each of the compositions for 28 days in volunteers with skin imperfections. In particular, an improvement was observed in 24% of volunteers after application of composition 1 and in 75% of volunteers after application of composition 2.

An evaluation of cosmetic acceptability by volunteers was also carried out after using composition 1 or 2 for 28 days. The results showed an overall satisfaction rate of 81% and 92% respectively and over 75% of volunteers confirmed the following properties of the composition:
The scent is pleasant
The texture is pleasant
The product is absorbed rapidly
The product is easy to apply
The product does not leave the skin sticky
The skin is soft
The skin is supple
The product improves the overall appearance of the skin
The product provides a feeling of freshness
The product leaves a protective film on the skin
The product provides protection against external aggressors
The product respects the skin
The product refines the texture of the skin
The product reduces skin shine
The product mattifies the skin
The product tightens pores
The product suits your skin type

This example clearly demonstrates that compositions comprising at least one substituted or unsubstituted polylysine dendrimer, at least one vitamin, for example vitamin A or vitamin C, and at least one cosmetically and/or dermatologically acceptable carrier cause no side effects after application to the skin and are non-irritating. In other words, this example clearly demonstrates that compositions comprising at least one substituted or unsubstituted polylysine dendrimer, at least one vitamin, for example vitamin A or vitamin C, and at least one cosmetically and/or dermatologically acceptable carrier have very good skin tolerance and compatibility.

### Example 14: Study of the Stability/Preservation of Compositions Comprising a Substituted or Unsubstituted Polylysine Dendrimer, at Least One Vitamin, for Example Vitamin a or Vitamin C, and at Least One Cosmetically and/or Dermatologically Acceptable Carrier

In this example, a study of the preservation of compositions in relation to bacterial strains was carried out. The compositions tested were compositions 1 and 2 mentioned hereinbefore. The assessment was carried out against the criteria and according to the protocol of ISO 11930.

The tested composition was divided into sterile vials and each vial was inoculated with a suspension of one of the test strains. Incubation was carried out at 22.5° C.±2.5° C. in the dark for 28 days. Neutralization validation was carried out on all strains. The preservative neutralization phase was carried out at the lowest dilution compatible with validation. The microbial densities obtained during monitoring for each of the strains tested were counted and compared with the logarithmic reduction thresholds imposed by the standard.

The strains used and the experimental conditions are summarized in Table 26.

TABLE 26
Experimental conditions

| | | Culture | Densities | Incubation |
|---|---|---|---|---|
| Strains | Preservation | media | (CFU/mL) | temperature |
| Pseudomonas | −80° C. ± 5° C. | Tryptic Soy | 10⁷-10⁸ | 32.5° C. ± 2.5° C. |
| aeruginosa ATCC | | Agar | | |
| 9027 | | | | |
| Staphylococcus | −80° C. ± 5° C. | Tryptic Soy | 10⁷-10⁸ | 32.5° C. ± 2.5° C. |
| aureus ATCC 6538 | | Agar | | |
| Escherichia coli | −80° C. ± 5° C. | Tryptic Soy | 10⁷-10⁸ | 32.5° C. ± 2.5° C. |
| ATCC 8739 | | Agar | | |
| Candida albicans | −80° C. ± 5° C. | Sabouraud | 10⁶-10⁷ | 27.5° C. ± 2.5° C. |
| ATCC 10231 | | Dextrose | | |
| Aspergillus | −80° C. ± 5° C. | Potato | 10⁶-10⁷ | 27.5° C. ± 2.5° C. |
| brasiliensis ATCC | | Extract Agar | | |
| 16404 | | | | |

The enumeration method was performed by inclusion with results given in CFU/g or mL.

The compliance criteria are summarized in Table 26 below.

TABLE 27
Conformity criteria according to ISO 11930

| | Bacteria | | | C. albicans | | | A. brasiliensis | |
|---|---|---|---|---|---|---|---|---|
| | D7 | D14 | D28 | D7 | D14 | D28 | D14 | D28 |
| Criteria A | ≥3 | ≥3 + NI | ≥3 + NI | ≥1 | ≥1 + NI | ≥1 + NI | ≥0 | ≥1 + NI |
| Criteria B | — | ≥3 | ≥3 + NI | — | ≥1 | ≥1 + NI | ≥0 | ≥0 + NI |

In Table 27, NI means no increase in the number of microorganisms compared with the previous contact time

The neutralizer was LT SUP Broth comprising casein pancreatic peptone 15 g/L, soybean papain peptone 5 g/L, L-Cystine 0.7 g/L, sodium chloride 4.0 g/L, sodium sulfite 0.2 g/L, glucose 5.5 g/L, egg lecithin 1.0 g/L, polysorbate 80 15.0 g/L and sodium laureth sulfate 1.56 g/L

Table 28 shows the results of bacterial density in composition 1 after inoculation in CFU/g.

TABLE 28
Bacterial density in composition 1

| | Pseudomonas | Staphylococcus | Escherichia | Candida | Aspergillus |
|---|---|---|---|---|---|
| | aeruginosa | aureus | coli | albicans | brasiliensis |
| Strains | (Pa) | (Sa) | (Ec) | (Ca) | (Ab) |
| Neutralization | Validated | Validated | Validated | Validated | Validated |
| D 0 | 1.8E+5 | 1.1E+5 | 1.6E+5 | 1.6E+4 | 3.0E+4 |
| D 7 | <10 | <10 | <10 | <10 | / |
| D 14 | <10 | <10 | <10 | <10 | <10 |
| D 28 | <10 | <10 | <10 | <10 | <10 |

Table 29 shows the interpretation of results obtained with composition 1 according to the criteria of ISO 11930

TABLE 29
Bacterial density in composition 1 using the 5 criteria of ISO 11930

| | Pseudomonas | Staphylococcus | Escherichia | Candida | Aspergillus |
|---|---|---|---|---|---|
| | aeruginosa | aureus | coli | albicans | brasiliensis |
| Strains | (Pa) | (Sa) | (Ec) | (Ca) | (Ab) |
| D 7 | >3 | >3 | >3 | >1 | / |
| D 14 | >3 | >3 | >3 | >1 | >1 |
| D 28 | >3 | >3 | >3 | >1 | >1 |
| Interpretation | A | A | A | A | A |

Table 30 shows the results of bacterial density in composition 2 after inoculation in CFU/g.

TABLE 30
Bacterial density in composition 2

| | Pseudomonas | Staphylococcus | Escherichia | Candida | Aspergillus |
|---|---|---|---|---|---|
| | aeruginosa | aureus | coli | albicans | brasiliensis |
| Strains | (Pa) | (Sa) | (Ec) | (Ca) | (Ab) |
| Neutralization | Validated | Validated | Validated | Validated | Validated |
| D 0 | 1.8E+5 | 1.1E+5 | 1.6E+5 | 1.6E+4 | 3.0E+4 |
| D 7 | <10 | <10 | <10 | <10 | 1 |
| D 14 | <10 | <10 | <10 | <10 | <10 |
| D 28 | <10 | <10 | <10 | <10 | <10 |

Table 31 shows the interpretation of results obtained with composition 2 using the criteria of ISO 11930

TABLE 31
Bacterial density in composition 2 using the criteria of ISO 11930

| | Pseudomonas | Staphylococcus | Escherichia | Candida | Aspergillus |
|---|---|---|---|---|---|
| | aeruginosa | aureus | coli | albicans | brasiliensis |
| Strains | (Pa) | (Sa) | (Ec) | (Ca) | (Ab) |
| D 7 | >3 | >3 | >3 | >1 | / |
| D 14 | >3 | >3 | >3 | >1 | >1 |
| D 28 | >3 | >3 | >3 | >1 | >1 |
| Interpretation | A | A | A | A | A |

As demonstrated in this example, compositions comprising a substituted or unsubstituted polylysine dendrimer, at least one vitamin, for example vitamin A or vitamin C, and at least one cosmetically and/or dermatologically acceptable carrier are stable over time and do not allow bacterial growth after inoculation of said composition. This example clearly demonstrates the effectiveness of antimicrobial preservatives.

### REFERENCES

1. WO2006114528A1
2. An Expeditious Multigram-Scale Synthesis of Lysine Dendrigraft (DGL) Polymers by Aqueous N-Carboxyanhydride Polycondensation. H. Collet and collaborators: Chem. Eur. J. 2010, 16, 2309-2316.
3. Fregert S. Manual of Contact Dermatitis. 2nd ed. Munksgaard, Copenhagen: Year Book Medical Publishers, Inc; 1981. Chapter 10, Patch Testing; pp. 71-81.
4. International Classification of Diseases (B07, ICD10, Chapter I),
5. International Classification of Diseases (L82, ICD10, Chapter XII),
6. International Classification of Diseases (L20-L30, ICD10, Chapter XII)
7. International Classification of Diseases (L73.2, ICD10, Chapter XII)
8. International Classification of Diseases (T23, ICD10, Chapter XIX)
9. International Classification of Diseases (L13.0, ICD10, Chapter XII)
10. Scholl, Markus, et al. “The thermal polymerization of amino acids revisited; Synthesis and structural characterization of hyperbranched polymers from l-lysine.” Journal of Polymer Science Part A: Polymer Chemistry 45.23 (2007): 5494-5508
11. Knowlton et Pearce [Knowlton, John L., and Steven EM Pearce.] Handbook of Cosmetic Science & Technology. Elsevier, 2013
12. European Cosmetics Association. “Cosmetics Europe: Guidelines on stability testing of cosmetic products. Guidel Stab Test Cosmet Prod [Internet]. Cosmetics Europe; 2004 March” and Romanowski, Perry, and Randy Schueller. “Stability testing of cosmetic products.” Novel Cosmetic Delivery Systems 6 (2023): 115-129.

1. A non-therapeutic cosmetic use of a cosmetic composition comprising at least one substituted or unsubstituted polylysine dendrimer, at least one vitamin and at least one cosmetically acceptable carrier.

2. The non-therapeutic cosmetic use according to claim 1, wherein said at least one substituted or unsubstituted polylysine dendrimer is a substituted or unsubstituted 2^{nd} generation (G2) to 10th generation (G10) polylysine dendrimer.

3. The non-therapeutic cosmetic use according to claim 1, wherein said at least one vitamin is selected from the group comprising water-soluble vitamins and fat-soluble vitamins.

4. The non-therapeutic cosmetic use according to claim 1 wherein said polylysine dendrimer is substituted by at least one group selected from aspartate, succinate, guanidyl, acetate or fluorine.

5. The non-therapeutic cosmetic use according to claim 1, wherein said cosmetic composition comprises from 0.01 to 10% by weight of said at least one vitamin with respect to the total weight of the composition.

6. The non-therapeutic cosmetic use according to claim 1, wherein said cosmetic composition comprises from 0.01 to 10% by weight of said at least one substituted or unsubstituted polylysine dendrimer with respect to the total weight of the composition.

7. The non-therapeutic cosmetic use according to claim 1, wherein said at least one vitamin is selected from the group comprising vitamin B1, vitamin B2, vitamin B3, vitamin B5, vitamin B8, vitamin B9, vitamin B12, vitamin C, vitamin A, vitamin D, vitamin E, vitamin K1 and vitamin K2, preferably selected from the group comprising vitamin C and vitamin A.

8. The non-therapeutic cosmetic use according to claim 1, wherein said composition is in a form selected from a gel, ointment, cream, oil, milk, ointment, powder, soaked pad, solution, serum, balm, butter, lotion, suspension, soap and emulsion.

9. A non-therapeutic cosmetic care method, comprising the application to the skin or mucous membranes of a cosmetic composition as defined according to claim 1.

10. The method according to claim 9, wherein the cosmetic care is selected from hydrating, improving the appearance and soothing the skin and/or its appendages and/or the mucous membranes.

11. A dermatological composition comprising at least one substituted or unsubstituted polylysine dendrimer, at least one vitamin and at least one dermatologically acceptable carrier for use in the treatment of a skin condition.

12. A composition for use according to claim 11 wherein the skin condition is selected from the group comprising skin inflammation and/or a skin infection.

13. The composition for use according to claim 11 wherein said at least one substituted or unsubstituted polylysine dendrimer is a substituted or unsubstituted 2^{nd} generation (G2) to 10^{th} generation (G10) polylysine dendrimer.

14. The composition for use according to claim 11, wherein said at least one vitamin is selected from the group comprising water-soluble vitamins and fat-soluble vitamins.

15. The composition for use according to claim 11 wherein the skin condition is selected from the group comprising actinic keratosis, impetigo, pemphigus, dermatitis herpetiformis, (L20) atopic dermatitis, seborrheic dermatitis, allergic contact dermatitis, irritant contact dermatitis, lichen simplex chronicus and prurigo, pruritus, psoriasis, lichen planus, urticaria, sunburn, radiation dermatitis, peladic dermatitis, onycholysis, alopecia, vitiligo, seborrheic keratosis, pressure sores, seborrheic wart, eczema, herpes, skin burn.
